(19) United States
(12) Patent Application Publication
(10) Pub. No.: US 2025/0332590 A1
(43) Pub. Date: Oct. 30, 2025

(54) MICROFLUIDIC DEVICE, SYSTEM, KIT, METHOD OF ANALYZING NUCLEIC ACIDS, METHOD OF MANIPULATING NUCLEIC ACIDS, METHOD OF DETECTING A BIOMOLECULE, AND METHOD OF ANALYZING A BIOMOLECULE

(71) Applicant: SINGLERON (NANJING) BIOTECHNOLOGIES, LTD., Nanjing (CN)

(72) Inventors: Yunyen CHIU, Suzhou (CN); Mengya QIAN, Nanjing (CN); Guoxin TANG, Nanjing (CN); Wenqi ZHU, Nanjing (CN); Yanhong MA, Suzhou (CN)

(21) Appl. No.: 18/866,970

(22) PCT Filed: May 16, 2023
(86) PCT No.: PCT/CN2023/094410

§ 371 Date: Nov. 18, 2024

(30) Foreign Application Priority Data
May 18, 2022 (WO) PCT/CN2022/093502

(51) Int. Cl. B01L 3/00; C12N 15/10; C12Q 1/6806; C12Q 1/6869

(52) U.S. Cl. CPC B01L 3/502715; B01L 3/502761; C12N 15/1013; C12Q 1/6806; C12Q 1/6869; B01L 2300/0645; B01L 2300/0816; B01L 2300/0829; B01L 2300/0887; B01L 2400/0421

(57) ABSTRACT

The present disclosure provides a microfluidic device, a system, a kit, a method of analyzing nucleic acids, a method of manipulating nucleic acids, a method of detecting a biomolecule, and a method of analyzing a biomolecule. The microfluidic device may include electrically conductive layers, to which an electric field can be applied to control movement of biomolecules associated with the single cell in the device. For example, polar molecules (e.g., RNA) of the cell can be captured (e.g. by barcode molecules) and analyzed with improved efficiency.

### FIELD

The present disclosure relates generally to the field of molecular biology, specifically to a microfluidic device, a system, a kit, a method of analyzing nucleic acids, a method of manipulating nucleic acids, a method of detecting a biomolecule, and a method of analyzing a biomolecule.

### DESCRIPTION OF THE RELATED ART

Microwell array devices have been used in single cell RNA sequencing. The cells can be lysed in a microwell to release RNA, which can then be captured (e.g., by beads) for sequencing. While the location of a single cell on a microwell array can be traced, RNA molecules are still mobile during the release and capture processes in such half closed system. The mobility of the released RNA molecules limits their capacity for further reaction in the microwell. Thus, there remains a need for devices with improved efficiency for single cell analysis.

### SUMMARY

Provided herein include devices, reagents, systems, kits, and methods for high-throughput single cell analysis using electrophoresis.

Disclosed herein include a microfluidic device. The can include a first layer with a lower surface comprising a first electrically conductive layer disposed thereon; a second layer with a microwell array disposed thereon and comprising at least 100 microwells. A upper surface of the array of microwells includes a second electrically conductive layer disposed thereon and not in contact with the first electrically conductive layer; a first electric terminal and a second electric terminal in electrical communication with the first electrically conductive layer and the second electrically conductive layer, respectively; a flow channel formed by the first layer and the second layer; and an inlet and an outlet in fluid communication with the flow channel. The first layer can be in direct contact with the second layer. The microfluidic device can include a third layer between the first layer and the second layer. For example, the third layer can be in direct contact with the first layer and the second layer.

In some embodiments, the first layer is a cover plate, and the second layer is a bottom plate. For example, the microfluidic device can include a cover plate and/or a bottom plate. The first electrically conductive layer can be, for example, rectangular in shape. The first electrically conductive layer can be about 5 cm² in size. About 50% of the lower surface of the first layer can include the first electrically conductive layer. The second electrically conductive layer can be, for example, rectangular in shape. The second electrically conductive layer can be about 5 cm² in size. About 50% of the upper surface of the second layer can include the second electrically conductive layer. In some embodiments, a shape of the first electrically conductive layer and a shape of the second electrically conductive layer can be identical. In some embodiments, a size of the lower surface of the first layer and a size of the upper surface of the second layer can be identical.

The first electric terminal can be on an outer surface of the microfluidic device. For example, the first electric terminal can extrude from, and/or be recessed into, an outer surface of the microfluidic device. In some embodiments, the second electric terminal can be on an outer surface of the microfluidic device. For example, the second electric terminal can extrude from, and/or be recessed into, an outer surface of the microfluidic device.

The microfluidic device can further include a first indicator that indicates that the first electric terminal is a negative electric terminal. In some embodiments, the microfluidic device can further include a second indicator that indicates that the second electric terminal is a positive electric terminal. The first indicator and/or the second indicator can be on an outer surface of the microfluidic device.

The flow channel can include a rectangular section. The flow channel can include a first tapered end, which can be, for example, triangular in shape. In some embodiments, the inlet is at the first tapered end. In some embodiments, the flow channel can include a second tapered end, which can be, for example, triangular in shape. In some embodiments, the outlet is at the second tapered end. In some embodiments, the inlet includes a hole in the first layer or the second layer, and/or the outlet includes a hole in the first layer or the second layer.

The flow channel includes an outer surface of the first electrically conductive layer. In some embodiments, the flow channel includes an outer surface of the second electrically conductive layer. In some embodiments, a distance between the first layer and the second layer is about 1 μm to about 100 μm.

In some embodiments, a thickness of the first electrically conductive layer is about 0.1 μm to about 5 μm. In some embodiments, a thickness of the second electrically conductive layer is about 0.1 μm to about 5 μm.

In some embodiments, a width of the microwell is 10 μm to 200 μm. In some embodiments, a length of the microwell is 10 μm to 200 μm. In some embodiments, a depth of the microwell is 5 μm to 500 μm. In some embodiments, the microwell has a circular, elliptical, square, rectangular, triangular, or hexagonal shape.

Disclosed herein include a system. The system can include a holder of a microfluidic device as described herein; an inlet fluidic interface for fluidic communication with the inlet of the microfluidic device; an outlet fluidic interface for fluidic communication with the outlet of the microfluidic device; one or more pumps for introducing one or more fluids into the microfluidic device via the inlet fluidic interface and the inlet of the microfluidic device; a first electric interface for connecting with the first electric terminal of the microfluidic device; and a second electric interface for connecting with the second electric terminal of the microfluidic device.

Disclosed herein include a kit. The kit can include a microfluidic device as described herein, and instructions for using the microfluidic device. In some embodiments, the instructions include instructions for applying a voltage between (i) the first electrical terminal as a negative terminal and (ii) the second electrical terminal as a positive terminal. In some embodiments, the instructions include instructions for using the microfluidic device for single cell sequencing. For example, the single cell sequencing includes single cell RNA sequencing or multiomics sequencing. The kit can also include one or more reagents for single cell sequencing.

Disclosed herein include methods of analyzing nucleic acids. In some embodiments, the method includes co-partitioning a plurality of cells and a plurality of particles into a plurality of microwells of a microwell array, thereby at least 25% of the plurality of microwells each includes a single cell of the plurality of cells and a single particle of the plurality of particles; applying an electric field to the microwell array; while applying the electric field to the microwell array: releasing a plurality of target nucleic acids associated with the single cell in a microwell of the plurality of microwells; and barcoding the plurality of target nucleic acids released to generate a plurality of barcoded nucleic acids; and analyzing the plurality of barcoded nucleic acids.

In some embodiments, the method of analyzing nucleic acids includes co-partitioning a plurality of cells and a plurality of particles into a plurality of microwells of a microwell array, thereby at least 25% of the plurality of microwells each includes a single cell of the plurality of cells and a single particle of the plurality of particles; releasing a plurality of target nucleic acids associated with the single cell in a microwell of the plurality of microwells; applying an electric field to the microwell array; while applying the electric field to the microwell, barcoding the plurality of target nucleic acids released to generate a plurality of barcoded nucleic acids; and analyzing the plurality of barcoded nucleic acids.

For example, the particles can each includes a plurality of barcode molecules, and barcoding the plurality of target nucleic acids released to generate a plurality of barcoded nucleic acids includes barcoding the plurality of target nucleic acids released using the plurality of barcode molecules of the particle in the microwell to generate a plurality of barcoded nucleic acids.

In some embodiments, the method of analyzing nucleic acids includes partitioning a plurality of cells into a plurality of microwells of a microwell array, thereby at least 25% of the plurality of microwells each includes a single cell of the plurality of cells; releasing a plurality of target nucleic acids associated with the single cell in a microwell of the plurality of microwells; applying an electric field to the microwell array; and while applying the electric field to the microwell: introducing a reaction reagent into the microwell; and performing a reaction on the target nucleic acids using the reaction reagent. In some embodiments, the reaction reagent can include one or more barcoding reagents, and the reaction includes a barcoding reaction. For example, the barcoding reagents can include barcode molecules. In some embodiments, the method can further include, after partitioning the plurality of cells into the plurality of microwells and prior to applying the electric field, partitioning a plurality of particles each comprising a plurality of barcode molecules into the plurality of microwells, thereby at least 25% of the plurality of microwells each includes a single cell of the plurality of cells and a single particle of the plurality of particles. In some embodiments, the method can further include analyzing a reaction product of the target nucleic acids generated using the reaction reagent.

Disclosed herein include a method of manipulating nucleic acids. The method can include partitioning a plurality of cells into a plurality of microwells, thereby at least 25% of the plurality of microwells each includes a single cell of the plurality of cells; releasing a plurality of target nucleic acids associated with the single cell in a microwell of the plurality of microwells; and applying an electric field to the microwell array, thereby controlling a movement of the target nucleic acids. In some embodiments, the method can further include, before releasing the plurality of target nucleic acids, partitioning a plurality of particles into the plurality of microwells, thereby at least 25% of the plurality of microwells each includes a single cell of the plurality of cells and a single particle of the plurality of particles.

Also provided include a method of analyzing nucleic acids. The method can include manipulating a plurality of target nucleic acids as describe herein, barcoding the plurality of target nucleic acids released using a plurality of barcode molecules to generate a plurality of barcoded nucleic acids; and analyzing the plurality of barcoded nucleic acids.

Disclosed herein include a method of detecting a biomolecule. The method can include, partitioning a plurality of cells into a plurality of microwells of a microwell array, thereby at least 25% of the plurality of microwells each includes a single cell of the plurality of cells; introducing a reaction reagent into the plurality of microwells; applying an electric field to the plurality of microwells, thereby the reaction reagent enters the cell; within the cell, the reaction reagent interacts with a biomolecule, and detecting the biomolecule. In some embodiments, the electric field increases a permeability of a membrane of the cell, thereby the reaction reagent enters the cell after the permeability of the membrane of the cell is increased. In some embodiments, the electric field causes electroporation. In some embodiments, the reaction reagent is a probe capable of capturing the biomolecule. The probe can be, for example, a small molecule compound, a polypeptide, an oligonucleotide, a ribonucleic acid (RNA), a deoxyribonucleic acid (DNA), an oligosaccharide, a sugar, or a combination thereof.

Disclosed herein include a method of analyzing a biomolecule. The method can include within a partition comprising a biomolecule, applying an electric field, thereby manipulating a movement of the biomolecule within the partition; and analyzing the biomolecule. The biomolecule used in the present methods can be, for example, a polypeptide, a protein, an oligonucleotide, a ribonucleic acid (RNA), a deoxyribonucleic acid (DNA), an oligosaccharide, a sugar, or a combination thereof. In some embodiments, the biomolecule is associated or previously associated with a single cell. For example, the method of analyzing a biomolecule can include introducing the single cell into the partition, and optionally the method can include releasing the biomolecule from the single cell. In some embodiments, the method further includes introducing a plurality of barcode molecules into the partition. In some embodiments, the method further includes barcoding the biomolecule using the plurality of barcode molecules. The partition can be, for example, a droplet or a microwell of a microwell array comprising a plurality of microwells. For example, the partition is a microwell of a microwell array comprising a plurality of microwells.

In some embodiments, applying the electric field to the microwell is performed before releasing the plurality of target nucleic acids associated with the single cell in the microwell of the plurality of microwells. In some embodiments, releasing the plurality of target nucleic acids includes: while applying the electric field to the microwell array, releasing the plurality of target nucleic acids associated with the single cell in the microwell of the plurality of microwells. In some embodiments, applying the electric field to the microwell is performed after releasing the plurality of target nucleic acids associated with the single cell in the microwell of the plurality of microwells.

In some embodiments, a method disclosed herein can further include while applying the electric field: introducing a first reaction reagent into the microwell; and performing a first reaction on a content of the single cell in the microwell using the first reaction reagent. In some embodiments, the method can further include while applying the electric field: introducing a second reaction reagent into the microwell; and performing a second reaction on a content of the single cell in the microwell using the second reaction reagent.

In some embodiments, each of the plurality of particles includes a plurality of barcode molecules. Each barcode molecule of the plurality of barcode molecules, for example, can include a molecular barcode sequence, a particle barcode sequence, and optionally a target binding sequence. In some embodiments, as a result of the partitioning, at least 90% of the plurality of microwells each includes at most one of the plurality of cells.

In some embodiments, the electric field restricts the movement the plurality of target nucleic acids or biomolecule in the partition. In some embodiments, the microwell includes an open end and a closed end facing the open end, the electric field is applied in a direction from the opened end to the closed end, or an opposite direction thereof.

In some embodiments, barcoding the plurality of target nucleic acids includes extending the plurality of barcode molecules using the plurality of target nucleic acids as templates to generate the plurality of barcoded nucleic acids comprising a plurality of single-stranded barcoded nucleic acids, optionally hybridized to the plurality of target nucleic acids. In some embodiments, the method further includes introducing a plurality of template switching oligonucleotides into the microwell, barcoding the plurality of target nucleic acids includes extending the plurality of barcode molecules using the plurality of target nucleic acids and the plurality of template switching oligonucleotides as templates to generate the plurality of barcoded nucleic acids comprising a plurality of single-stranded barcoded nucleic acids.

In some embodiments, the method further includes introducing a plurality of extension primers to the microwell, and barcoding the plurality of target nucleic acids includes extending the plurality of extension primers using the plurality of target nucleic acids as templates and the plurality of barcode molecules as template switching oligonucleotides to generate the plurality of barcoded nucleic acids comprising a plurality of single-stranded barcoded nucleic acids.

Each of the plurality of single-stranded barcoded nucleic acids can be, for example, hybridized to one of the plurality of target nucleic acids and one of the plurality of template switching oligonucleotides in the microwell. In some embodiments, the method further includes removing the plurality of target nucleic acids and the plurality of template switching oligonucleotides hybridized to the single-stranded barcoded nucleic acids. Removing the plurality of target nucleic acids can include, for example, denaturation, thermal denaturation, digesting, or hydrolyzing the plurality of target nucleic acids. Each of the plurality of single-stranded barcoded nucleic acid can include, for example, a sequence of a barcode molecule of the plurality of barcode molecules, a sequence of a target nucleic acid of the plurality of target nucleic acids, a sequence of a template switching oligonucleotide of the plurality of template switching oligonucleotides, and/or a sequence of an extension primer of the plurality of extension primers.

In some embodiments, the method further includes amplifying the plurality of barcoded nucleic acids to generate a plurality of double-stranded barcoded nucleic acids in the microwell using the single-stranded barcoded nucleic acids as templates.

In some embodiments, the plurality of target nucleic acids includes poly-adenylated messenger ribonucleic acid (mRNA) and the extension primers include a poly(dT) sequence.

In some embodiments, each of the plurality of barcode molecules includes a primer sequence. For example, the primer sequence can include a PCR primer sequence. Amplifying the plurality of barcoded nucleic acids can include amplifying the plurality of barcoded nucleic acids using the primer sequences in single-stranded barcoded nucleic acids of the plurality of single-stranded barcoded nucleic acids, or products thereof.

The plurality of target nucleic acids can include, for example, deoxyribonucleic acid (DNA) or ribonucleic acid (RNA). Barcoding the plurality of target nucleic acids can include, for example, a reverse transcription reaction, and the plurality of barcoded nucleic acids includes complementary deoxyribonucleic acid (cDNA). In some embodiments, barcoding the plurality of target nucleic acids includes hybridizing the target binding sequence to a target nucleic acid of the plurality of target nucleic acids. The target binding sequence can include, for example, a poly(dT) sequence and/or a sequence capable of hybridizing to the target nucleic acid, optionally the sequence includes a target specific sequence. For example, the target binding sequence of the barcode molecule can include a poly(dT) sequence, and barcoding the plurality of target nucleic acids can include hybridizing the poly(dT) sequence of the target binding sequence to a poly(A) sequence of a target nucleic acid of the plurality of target nucleic acids.

Releasing the plurality of target nucleic acids associated with the single cell in a microwell of the plurality of microwells can include, for example, lysing the single cell using a lysis agent.

In some embodiments, the plurality of barcode molecules are attached to, reversibly attached to, covalently attached to, or irreversibly attached to the particle. In some embodiments, the particle is a bead. In some embodiments, the particle is a gel particle, such as a hydrogel particle. In some embodiments, the gel particle is degradable upon application of a stimulus. The stimulus, for example, can include a thermal stimulus, a chemical stimulus, a biological stimulus, a photo-stimulus, or a combination thereof. In some embodiments, the particle is a solid particle and/or a magnetic particle. For example, the particle can be retained in the microwell by an external magnetic field. In some embodiments, the particle includes a paramagnetic material. In some embodiments, the particle has a size of about 10 μm to about 100 μm.

The molecular barcode sequence can include unique molecule identifiers (UMIs). The UMIs, for example, can be 2-40 nucleotides in length. In some embodiments, the particle barcode sequences of the plurality of barcode molecules on a single particle are identical. In some embodiments, each of the plurality of barcode molecules includes a primer sequence. The primer sequence, for example, can be a sequencing primer sequence, such as a Read 1 sequence, a Read 2 sequence, or a portion thereof. In some embodiments, a barcode molecule of the plurality of barcode molecules includes a template switching oligonucleotide. In some embodiments, analyzing the plurality of barcoded nucleic acids, or products thereof, includes determine the sequences of the plurality of barcoded nucleic acids, or products thereof.

Disclosed herein include a microfluidic device, and method of use thereof, for single cell analysis, in which electric potentials are applied to a microwell array in a direction perpendicular to the microwell array (e.g., the direction from the opening to the bottom of the microwell). For example, the surface of the microwells can be positively charged, and a surface of a flow channel opposite to the microwells can be negatively charged. After the device is electrically charged, movement of RNAs released from the cell are restricted in the microwell by the positively charged surface of the microwell (e.g., by electrophoresis), thereby the RNAs are captured by a bead (e.g., through hybridizing with barcode molecules attached to the bead) in the microwell. The present microfluidic device and method also can be used to conduct various reagent exchange reactions (e.g., reverse transcription, amplification) while RNA is restricted (or immobilized) in the microwell by the positive electric potential. Further, electroporation can be performed by increasing the applied electric potentials (e.g., causing an opening of the cell membrane), so that a probe can be introduced to capture molecules of interest (e.g., RNA) inside the cell. In addition to microwell array-based devices, the immobilization of polar molecules by an electric field also can be used in other microfluidic devices (e.g., droplet-based devices) and methods.

Advantageously, the present microfluidic device and method can improve efficiency of polar molecule (e.g., RNA, DNA, protein) capture for single cell analysis, enable reagent exchange reactions (e.g., with the polar molecules immobilized in the microwell), and improve mixing efficiency of different materials in the microwell (e.g., by adjusting the polarity and strength of the electric field), thereby improving reaction efficiency.

### BRIEF DESCRIPTION OF THE DRAWINGS

FIGS. 1A and 1B show non-limiting embodiments of microfluidic chips disclosed herein (1: sample inlet; 2: flow channel; 3: lower plate; 4: upper plate; 5: microwell array area; 6: sample outlet). FIG. 1C shows a non-limiting workflow of sample preparation as disclosed herein.

FIGS. 2A-2C show a non-limiting design of a microfluidic chip disclosed herein. FIG. 2A shows a upper layer of the chip, in which an electrically conductive layer (shaded area, e.g., negatively charged) covers a flow channel with tapered ends. FIG. 2B shows a lower layer of the chip, in which an electrically conductive layer (shaded area, e.g., positively charged) covers a microwell array in a rectangular area. FIG. 2C shows a top view and a corresponding cross section view of the chip.

FIGS. 3A and 3B shows the results of analyses of cells from mouse testicle. FIG. 3A shows the result using a control device. FIG. 3B shows the result using the present microfluidic chip with applied electric potentials (1.6V, 15 min).

FIG. 4 shows clusters of cells from mouse testicle identified using the present microfluidic chip.

FIG. 5 shows a comparison of the results obtained in the presence (Exp) and absence (Control) of applied electric field for the analysis of cells from mouse testicle.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Provided include devices, reagents, systems, kits, and methods for performing high-throughput single cell analysis using electrophoresis.

### Microfluidic Device

Disclosed herein include embodiments of a microfluidic device. The microfluidic device can be in a form of a microfluidic chip (such as a disposable chip). The microfluidic device can include a first layer with a lower surface comprising a first electrically conductive layer disposed thereon. The lower surface, for example, can be a bottom surface of the first layer. The microfluidic device can include a second layer with a microwell array disposed thereon and comprising, for example, at least 100 microwells. A upper surface of the array of microwells can include a second electrically conductive layer disposed thereon. In some embodiments, the second electrically conductive layer is not in contact, directly or indirectly, with the first electrically conductive layer. The upper surface, for example, can be a top surface of the second layer. The microfluidic device can include a first electric terminal and a second electric terminal in electrical communication with the first electrically conductive layer and the second electrically conductive layer, respectively. The first and second electric terminals, for example, can be connected with the first and second electronically conductive layers, respectively. The microfluidic device can include a flow channel formed by the first layer and the second layer. The microfluidic device can further include an inlet and an outlet in fluid communication with the flow channel.

In some embodiments, the first layer is in direct contact with the second layer. For example, a portion of the lower surface of the first layer, which does not include the first electrically conductive layer, can be in direct contact with a portion of the upper surface of the second layer, which does not include the second electrically conductive layer. The portions of the first layer and second layer that are in direct contact can be, for example, on peripheral parts (such as on one or more edges or sides) of the first layer and/or the second layer. The microfluidic device can include one or more additional layers, for example, on a side of the first layer, on a side of the second layer, or between the first layer and second layer. In some embodiments, the microfluidic device includes a third layer between the first layer and the second layer. The third layer, for example, can be in direct contact with the first layer and the second layer. In some embodiments, the third layer is a silicone pad. The layers of the microfluidic device can be connected with, attached to, coupled to, or bound to each other by any suitable means. For example, the layers can be bound together by an adhesive or by a screw clamp. In some embodiments, a through hole is provided in the layers, and the layers are connected by means of the through hole.

In some embodiments, the first layer is a cover plate (e.g., a upper plate or a top plate). In some embodiments, the second layer is a bottom plate (e.g., a lower plate). In some embodiments, the microfluidic device includes a cover plate and/or a bottom plate.

The first electrically conductive layer, for example, can have a circular, elliptical, square, rectangular, triangular, or hexagonal shape, or combinations thereof. In some embodiments, the first electrically conductive layer is rectangular in shape. As disclosed herein, a size can be, for example, area, width, length, depth (or height), radius, diameter, or circumference. The size (or area) of the first electrically conductive layer can be, be about, be at least, be at least about, be at most, or be at most about 0.01 cm², 0.05 cm², 0.1 cm². 0.5 cm², 1 cm², 2 cm², 3 cm², cm², 5 cm², 6 cm², 7 cm², 8 cm², 9 cm², 10 cm², 20 cm², 30 cm², 40 cm², 50 cm², 60 cm², 70 cm², 80 cm², 90 cm², 100 cm², 200 cm², 300 cm², 400 cm², 500 cm², 600 cm², 700 cm², 800 cm², 900 cm², 1000 cm², 2000 cm², 3000 cm², 4000 cm², 5000 cm², 6000 cm², 7000 cm², 8000 cm², 9000 cm², 10000 cm², or a number or a range between any two of these values. In some embodiments, the first electrically conductive layer is about 5 cm² in size.

In some embodiments, the lower surface of the first layer can be covered, at least in part, with the first electrically conductive layer. For example, the first electrically conductive layer can be deposited, and/or form a coating, on the lower surface of the first layer. The first electrically conductive layer can have an outer surface, which can, for example, face the second electrically conductive layer. The percentage of the lower surface of the first layer comprising the first electrically conductive layer can be, be about, be at least, be at least about, be at most, or be at most about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, about 50% of the lower surface of the first layer includes the first electrically conductive layer.

A thickness of the first electrically conductive layer can vary in different embodiments. For example, a thickness of the first electrically conductive layer can be, be about, be at least, be at least about, be at most, or be at most about, 0.01 μm, 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 600 μm, 700 μm, 800 μm, 900 μm, 1000 μm, or a number or a range between any two of these values. In some embodiments, a thickness of the first electrically conductive layer is about 0.1 μm to about 5 μm.

The second electrically conductive layer, for example, can have a circular, elliptical, square, rectangular, triangular, or hexagonal shape, or combinations thereof. In some embodiments, the second electrically conductive layer is rectangular in shape. The size (or area) of the second electrically conductive layer can be, be about, be at least, be at least about, be at most, or be at most about 0.01 cm², 0.05 cm², 0.1 cm², 0.5 cm², 1 cm², 2 cm², 3 cm², cm², 5 cm², 6 cm², 7 cm², 8 cm², 9 cm², 10 cm², 20 cm², 30 cm², 40 cm², 50 cm², 60 cm², 70 cm², 80 cm², 90 cm², 100 cm², 200 cm², 300 cm², 400 cm², 500 cm², 600 cm², 700 cm², 800 cm², 900 cm², 1000 cm², 2000 cm², 3000 cm², 4000 cm², 5000 cm², 6000 cm², 7000 cm², 8000 cm², 9000 cm², 10000 cm², or a number or a range between any two of these values. In some embodiments, the second electrically conductive layer is about 5 cm² in size.

In some embodiments, the upper surface of the second layer can be covered, at least in part, with the second electrically conductive layer. For example, the second electrically conductive layer can be deposited, and/or form a coating, on the upper surface of the second layer. The second electrically conductive layer can have an outer surface, which can, for example, face the first electrically conductive layer. The percentage of the upper surface of the second layer comprising the second electrically conductive layer can be, be about, be at least, be at least about, be at most, or be at most about 5%, 6%, 7%, 8%, 9%, 10%, 11%, 12%, 13%, 14%), 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, about 50% of the upper surface of the second layer includes the second electrically conductive layer.

A thickness of the second electrically conductive layer can vary in different embodiments. For example, a thickness of the second electrically conductive layer can be, be about, be at least, be at least about, be at most, or be at most about, 0.01 μm, 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 600 μm, 700 μm, 800 μm, 900 μm, 1000 μm, or a number or a range between any two of these values. In some embodiments, a thickness of the second electrically conductive layer is about 0.1 μm to about 5 μm.

The shapes of the first and second electrically conductive layers can be identical or different. In some embodiments, the shape of the first electrically conductive layer and the shape of the second electrically conductive layer are identical. The size (e.g., area) of the lower surface of the first layer and the size (e.g., area) of the upper surface of the second layer can be identical or different. In some embodiments, the size of the lower surface of the first layer and the size of the upper surface of the second layer are identical.

In some embodiments, the first electric terminal is on an outer surface (e.g., a top surface or side surface) of the microfluidic device. For example, the first electric terminal can extrude from an outer surface of the microfluidic device. The first electric terminal, for example, can be recessed into an outer surface of the microfluidic device. In some embodiments, the second electric terminal is on an outer surface (e.g., a top surface or side surface) of the microfluidic device. For example, the second electric terminal can extrude from an outer surface of the microfluidic device. The second electric terminal, for example, can be recessed into an outer surface of the microfluidic device. The first and second electric terminals can be connected to an electric power supply during the operation of the microfluidic device.

The microfluidic device can further include a first indicator that indicates that the first electric terminal is a negative (or positive) electric terminal. In some embodiments, the microfluidic device further includes a second indicator that indicates that the second electric terminal is a positive (or negative) electric terminal. The first and second indicators each can be on an outer surface (e.g., a top surface or side surface) of the microfluidic device. The first and second indicators can indicate the status (e.g., positive, negative, or neutral) of the first and second electric terminals, respectively, during the operation of the microfluidic device.

The flow channel can be formed, for example, by the lower surface of the first layer and the upper surface of the second layer. In some embodiments, the flow channel includes the outer surface of the first electrically conductive layer (e.g., facing the second electrically conductive layer). In some embodiments, the flow channel includes an outer surface of the second electrically conductive layer (e.g., facing the first electrically conductive layer). In some embodiments, the flow channel is defined, at least in part, by the outer surface of the first electrically conductive layer and the outer surface of the second electrically conductive layer.

The flow channel can have a circular, elliptical, square, rectangular, triangular, or hexagonal shape, or combinations thereof. The flow channel can have one more sections, and each section can have identical or different shapes. In some embodiments, the flow channel includes a rectangular section. The flow channel can have a cross section, which can have a circular, elliptical, square, rectangular, triangular, or hexagonal shape, or combinations thereof. In some embodiments, a cross section of the flow channel has a rectangular shape. A size (e.g., width, length, depth, height, radius, diameter, or circumference) of the flow channel can be, be about, be at least, be at least about, be at most, or be at most about, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 600 μm, 700 μm, 800 μm, 900 μm, 1000 μm (0.1 cm), 0.2 cm, 0.3 cm, 0.4 cm, 0.5 cm, 0.6 cm, 0.7 cm, 0.8 cm, 0.9 cm, 1 cm, 2, cm, 3 cm, 4 cm, 5 cm, 6 cm, 7 cm, 8 cm, 9 cm, 10 cm, 20 cm, 30 cm, 40 cm, 50 cm, 60 cm, 70 cm, 80 cm, 90 cm, 100 cm, or a number or a range between any two of these values. In some embodiments, a width or length of the flow channel can be 0.1 cm to 10 cm. In some embodiments, a diameter or height of the flow channel can be 1 μm to 100 μm.

The microfluidic device can have one or more additional flow channels. The additional flow channels can be used, for example, for sample loading, reagent loading, reagent mixing, sample/reagent transfer, waste removal, product isolation, and combinations thereof. The additional flow channels can be formed by known techniques including, but not limited to, casting, drilling, additive manufacturing, and the like, in one or more of the layers. The additional flow channels can also be formed using closed grooves between different layers. For example, matching grooves can be provided on the opposing surfaces of two layers, which can then be closed to form channels when the two layers are attached to each other (e.g., by lamination). A size (e.g., width, length, depth, height, radius, diameter, or circumference) of each of the additional flow channels (if present) can be, be about, be at least, be at least about, be at most, or be at most about, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 600 μm, 700 μm, 800 μm, 900 μm, 1000 μm (0.1 cm), 0.2 cm, 0.3 cm, 0.4 cm, 0.5 cm, 0.6 cm, 0.7 cm, 0.8 cm, 0.9 cm, 1 cm, 2, cm, 3 cm, 4 cm, 5 cm, 6 cm, 7 cm, 8 cm, 9 cm, 10 cm, 20 cm, or a number or a range between any two of these values. One, more than one, or each of the additional flow channels can be a microchannel. The additional flow channels (or microchannels) can form a network of channels.

The flow channel can include one or more tapered ends. Each of the tapered end can have a circular, elliptical, square, rectangular, triangular, or hexagonal shape. The shapes of different tapered ends can be same or different. For example, the flow channel can include a first tapered end. The first tapered end can be triangular in shape. The flow channel can also include a second tapered end. The second tapered end can be triangular in shape.

The inlet can used to load, inject, or otherwise introduce a cell, a material (e.g., a particle), and/or a reagent in a fluid (e.g., in an aqueous fluid) into the microfluidic device. The inlet can be directly connected to the flow channel. The inlet can be connected to the flow channel through one or more additional flow channels or a network of additional flow channels (or microchannels). In some embodiments, the inlet is at the first tapered end of the flow channel. The inlet can include a hole in the first layer or the second layer. The inlet can have a circular, elliptical, square, rectangular, triangular, or hexagonal shape. For example, the inlet can be a circular hole on an outer surface of the first layer and located at the first taper end of the flow channel, and the inlet is directly connected to the flow channel.

The outlet can used to remove or transfer a reagent, a waste, or a product (e.g., from a reaction) in a fluid (e.g., in an aqueous fluid) away from the microfluidic device, or from one part of the microfluidic device to another part of the microfluidic device. The outlet can be directly connected to the flow channel. The outlet can be connected to the flow channel through one or more additional flow channels or a network of additional flow channels (or microchannels). In some embodiments, the outlet is at the second tapered end of the flow channel. The outlet can include a hole in the first layer or the second layer. The outlet can have a circular, elliptical, square, rectangular, triangular, or hexagonal shape. For example, the outlet can be a circular hole on an outer surface of the first layer and located at the second taper end of the flow channel, and the outlet is directly connected to the flow channel.

A distance between the first layer and the second layer can vary in different embodiments. The distance between the first layer and the second layer can include, for example, the distance between the outer surface of the first electrically conductive layer and the outer surface of the second electrically conductive layer. For example, a distance between the first layer and the second layer can be, be about, be at least, be at least about, be at most, or be at most about, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 m, 600 μm, 700 μm, 800 μm, 900 μm, 1000 μm, 2000 μm, 3000 μm, 4000 μm, 5000 μm, or a number or a range between any two of these values. In some embodiments, a distance between the first layer and the second layer is about 1 μm to about 100 μm.

### System and Kit

Disclosed herein include embodiments of a system. The system can include a holder of a microfluidic device as described herein. The system can include an inlet fluidic interface for fluidic communication with the inlet of the microfluidic device. The system can include an outlet fluidic interface for fluidic communication with the outlet of the microfluidic device. The system can include one or more pumps for introducing one or more fluids into the microfluidic device via the inlet fluidic interface and the inlet of the microfluidic device. The system can include a first electric interface for connecting with the first electric terminal of the microfluidic device. The system can also include a second electric interface for connecting with the second electric terminal of the microfluidic device.

The holder can include, for example, a platform, a frame, a container, a housing, or the like. The holder can be capable of being coupled to, attached to, or connected to, the microfluidic device. The microfluidic device can be operated inside, on a surface of, and/or through connection with, the holder.

The inlet fluidic interface can include, for example, an inlet flow channel for injecting, loading, or transferring one or more fluids (e.g., an entering fluid) comprising a sample, a cell, a material (e.g., a particle), a reagent, or combinations thereof into the microfluidic device through the inlet of the microfluidic device. The inlet fluidic interface can include an inlet portal (e.g. a gate, an opening, or the like) for controlling the entry of the one or more entering fluids into the microfluidic device.

The outlet fluidic interface can include, for example, an outlet flow channel for removing or transferring one or more fluids (e.g., an exiting fluid) comprising a waste, a product, a material (e.g., a particle), or combinations thereof from the microfluidic device through the outlet of the microfluidic device. The outlet fluidic interface can include an outlet portal (e.g. a gate, an opening, or the like) for controlling the removal of the one or more exiting fluids from the microfluidic device.

The inlet flow channel of the inlet fluidic interface and the outlet flow channel of the outlet fluidic interface can be separate or distinct from the flow channel or the additional flow channels of the microfluidic device as disclosed herein. The inlet flow channel of the inlet fluidic interface or the outlet flow channel of the outlet fluidic interface can each include one, more than one, or a network of channels (or microchannels). A size (e.g., width, length, depth, height, radius, diameter, or circumference) of the inlet flow channel or the outlet flow channel can be, be about, be at least, be at least about, be at most, or be at most about, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 600 μm, 700 μm, 800 μm, 900 μm, 1000 μm (0.1 cm), 0.2 cm, 0.3 cm, 0.4 cm, 0.5 cm, 0.6 cm, 0.7 cm, 0.8 cm, 0.9 cm, 1 cm, 2, cm, 3 cm, 4 cm, 5 cm, 6 cm, 7 cm, 8 cm, 9 cm, 10 cm, 20 cm, 30 cm, 40 cm, 50 cm, 60 cm, 70 cm, 80 cm, 90 cm, 100 cm, or a number or a range between any two of these values.

The fluids, for example, can include an aqueous fluid, such as water, an aqueous solution, an aqueous suspension, or an aqueous buffer. In some embodiments, the fluid includes an aqueous solution or suspension of a reagent, an analyte, a cell, a waste, a product, of a combination thereof. Examples of aqueous buffer include, but are not limited to, Tris-HCl buffer, EDTA buffer, PBS buffer, HEPES buffer, MOPS buffer, MES buffer, citrate buffer, acetate buffer, phosphate buffer, and combinations thereof.

The one or more pumps can be, for example, attached to the inlet flow channel or the inlet portal. The pumps can be a pump assembly. The pump can be, for example, a gas pump configured to control gas pressure in the inlet flow channel or at the inlet portal, thereby injecting or introducing the one or more fluids into the microfluidic device.

The first and second electric interfaces each can include one or more plugs, connectors, adapters, fittings, wires, or combinations thereof for connecting with the electric terminals of the microfluidic device. The first and second electric interfaces can provide electric currents (direct current (DC) or alternating current (AC)), electric potentials (or voltages), electric signals, or a combination thereof to the first and second electric terminals the microfluidic device, respectively. The first and second electric interfaces can be connected to a power supply (such as a battery) and/or a control unit.

The system can also include a control unit for controlling the operation of the microfluidic device. The control unit, for example, can includes a control unit interface for controlling and/or programming the control unit, for example, using a computer, a control software, a programmable software, or a combination thereof. The system can include the computer.

Disclosed herein include embodiments of a kit. The kit can include a microfluidic device as described herein and instructions for using the microfluidic device. The instructions can include instructions for applying an electric field to the microfluidic device. The electric field can be applied to the first electric terminal and a second electric terminal as opposite terminals. As a result, an electric potential (voltage) can be formed between the first and second electric terminals. In some embodiments, the first electric terminal is a negative terminal (e.g., negatively charged) and the second electric terminal is a positive terminal (e.g., positively charged). In some embodiments, the first electric terminal is a positive terminal (e g., positively charged) and the second electric terminal is a negative terminal (e.g., negatively charged). For example, the instructions can include instructions for applying a voltage between (i) the first electrical terminal as a negative terminal and (ii) the second electrical terminal as a positive terminal.

The present systems and kits can be used for single cell analysis, including analysis of biomolecules (such as nucleic acids) associated with a single cell. The analysis can include, for example, sequencing nucleic acids associated with a single cell. In some embodiments, the instructions include instructions for using the microfluidic device for single cell sequencing. The sequencing can include, for example, DNA (genome) sequencing, DNA methylation (DNA methylome) sequencing, RNA (transcriptome) sequencing, transposase-accessible chromatin sequencing, and a combination thereof. In some embodiments, the single cell sequencing includes single cell RNA sequencing or multiomics sequencing.

In some embodiments, the kit includes one or more materials for cell analysis using the present microfluidic device. For example, the kit can further include one or more reagents for single cell sequencing, including, but not limited to, cell lysis agents, enzymes (such as reverse transcriptase, polymerase), chemical reagents, and particles (e.g., beads) each comprising a plurality of the barcode molecules. The kit can further include a plurality of cells. For example, the kit can include, include about, include at least, include at least about, include at most, or include at most about, 1000, 2000, 3000, 4000, 5000, 6000, 7000, 8000, 9000, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 5×10⁶, 1×10⁷, 5×10⁷, 1×10⁸, 5×10⁸, 1×10⁹, 5×10⁹, or 1×10¹⁰ cells.

### Methods

The present disclosure provides methods for manipulating, detecting, or analyzing biomolecules, such as biomolecules associated with a cell. The methods can include partitioning a plurality of cells into a plurality of partitions (e.g., microwells). The biomolecule can be inside or attached to the surface of the cell. The biomolecule can be a natural product of the cell or a synthetic molecule associated with the cell. The biomolecule, for example, can be a nucleic acid associated with the cell.

### Nucleic Acid Analysis

Disclosed herein include methods of analyzing nucleic acids. In some embodiments, the method includes co-partitioning a plurality of cells and a plurality of particles (e.g., beads) into a plurality of microwells of a microwell array, thereby, for example, at least 25% of the plurality of microwells each includes a single cell of the plurality of cells and a single particle of the plurality of particles. The method can include applying an electric field to the microwell array. While applying the electric field to the microwell array, the method can include releasing a plurality of target nucleic acids associated with the single cell in a microwell of the plurality of microwells; and barcoding the plurality of target nucleic acids released to generate a plurality of barcoded nucleic acids. The method can further include analyzing the plurality of barcoded nucleic acids.

In some embodiments, the method of analyzing nucleic acids includes co-partitioning a plurality of cells and a plurality of particles (e.g., beads) into a plurality of microwells of a microwell array, thereby, for example, at least 25% of the plurality of microwells each includes a single cell of the plurality of cells and a single particle of the plurality of particles. The method can include releasing a plurality of target nucleic acids associated with the single cell in a microwell of the plurality of microwells. The method can include applying an electric field to the microwell array. While applying the electric field to the microwell, the method can include barcoding the plurality of target nucleic acids released to generate a plurality of barcoded nucleic acids. The method can further include analyzing the plurality of barcoded nucleic acids.

In some embodiments, the particles each includes a plurality of barcode molecules. For example, barcoding the plurality of target nucleic acids released to generate a plurality of barcoded nucleic acids can include barcoding the plurality of target nucleic acids released using the plurality of barcode molecules of the particle in the microwell to generate a plurality of barcoded nucleic acids.

In some embodiments, the method of analyzing nucleic acids includes partitioning a plurality of cells into a plurality of microwells of a microwell array, thereby, for example, at least 25% of the plurality of microwells each includes a single cell of the plurality of cells. The method can include releasing a plurality of target nucleic acids associated with the single cell in a microwell of the plurality of microwells. The method can include applying an electric field to the microwell array. While applying the electric field to the microwell, the method can include introducing a reaction reagent into the microwell; and performing a reaction on the target nucleic acids using the reaction reagent.

The reaction reagent, as disclosed herein, can include any agent used in a cellular reaction. The reaction reagent can include, for example, a reagent capable of reacting with, binding to, or forming a covalent bond with a biomolecule. The reaction reagent can include an enzyme capable of catalyzing a reaction. In some embodiments, the reaction reagent can include one or more barcoding reagents, including, but not limited to a lysis buffer, restriction enzymes, ligase, polymerase, fluorophores, oligonucleotide barcodes, oligonucleotide probes, adapters, buffers, dNTPs, ddNTPs, and combinations thereof. In some embodiments, the reaction includes a barcoding reaction. In some embodiments, the barcoding reagents include barcode molecules (such as oligonucleotide barcode molecules). The barcode molecule can be introduced, for example, by introducing a plurality of particles each comprising a plurality of the barcode molecules.

The method can further include, after partitioning the plurality of cells into the plurality of microwells and prior to applying the electric field, partitioning a plurality of particles each comprising a plurality of barcode molecules into the plurality of microwells, thereby, for example, at least 25% of the plurality of microwells each includes a single cell of the plurality of cells and a single particle of the plurality of particles. The method can further include analyzing a reaction product of the target nucleic acids generated using the reaction reagent. The reaction product of the target nucleic acids can include, for example, barcoded nucleic acids generated from a barcoding reaction using the one or more barcoding reagents, such as the plurality of barcode molecules introduced by the particle. In some embodiments, the reaction product of the target nucleic acids includes a plurality of barcoded nucleic acids generated from a barcoding reaction using the plurality of barcode molecules of the particle. Analyzing the reaction product can include, for example, analyzing the plurality of barcoded nucleic acids.

### Biomolecule Manipulation and Analysis

Disclosed herein include methods of manipulating biomolecules, such as a poler or electronically charged biomolecule (e.g., nucleic acids) associated with a cell. In some embodiments, the method of manipulating nucleic acids includes partitioning a plurality of cells into a plurality of microwells, thereby, for example, at least 25% of the plurality of microwells each includes a single cell of the plurality of cells. The method can include releasing a plurality of target nucleic acids associated with the single cell in a microwell of the plurality of microwells. The method can further include applying an electric field to the microwell array, thereby controlling a movement of the target nucleic acids.

The electric field can be applied, for example, in a direction from an open end to an closed end (e.g., bottom) of the microwell, or the opposite direction. As a result, an electrophoresis can occur in the microwell, in which an electronically charged molecule can only move in the direction of the applied electric field. Movement of the electronically charged molecule in other directions can thus be restricted by the electric field. Nucleic acids can be negatively charged and, as a result, can be attracted to a positive potential of the electric field. For example, the microwell array can receive a positive potential of the electric field, thereby restricting the movement of the target nucleic acid to within the microwell and/or attracting the target nucleic acid the toward the bottom of the microwell. In some embodiments, the target nucleic acid is immobilized in the microwell by the applied electric field.

Controlling the movement of the target nucleic acid can facilitate the analysis of the target nucleic acid (e.g., by barcoding reaction using barcoding molecules). In some embodiments, the method can further include, before releasing the plurality of target nucleic acids, partitioning a plurality of particles into the plurality of microwells, thereby, for example, at least 25% of the plurality of microwells each includes a single cell of the plurality of cells and a single particle of the plurality of particles. In some embodiments, the method further includes, after releasing the plurality of target nucleic acids, partitioning a plurality of beads into a plurality of partitions, thereby, for example, at least 25% of the plurality of partitions each includes released target nucleic acids from the single cell and a single particle of the plurality of particles. The particles can each include a plurality of barcode molecules for performing a barcoding reaction.

In some embodiments, a method of analyzing nucleic acid is provide, which includes manipulating a plurality of target nucleic acids according the method described herein, barcoding the plurality of target nucleic acids released using a plurality of barcode molecules to generate a plurality of barcoded target nucleic acids, and analyzing the plurality of barcoded nucleic acids. The barcode molecules can be introduced, for example, by partitioning a plurality of particles each comprising a plurality of barcode molecules into the plurality of microwells as described herein.

Disclosed herein include methods of detecting a biomolecule. The method can include partitioning a plurality of cells into a plurality of microwells of a microwell array, thereby at least 25% of the plurality of microwells each includes a single cell of the plurality of cells. The method can include introducing a reaction reagent into the plurality of microwells. The method can include applying an electric field to the plurality of microwells, thereby the reaction reagent enters the cell. The reaction reagent can interacts with a biomolecule within the cell. The method can further include detecting the biomolecule.

In some embodiments, the electric field increases a permeability of a membrane of the cell, thereby the reaction reagent enters the cell after the permeability of the membrane of the cell is increased. In some embodiments, the electric field causes electroporation. Electroporation can result from transient changes in the permeability and electrical conductivity in the plasma and nuclear membranes of a cell, when a pulse with short time and high electrical field is applied to a cell. An accumulation of charges due to ions migration can occur when the electric field is being applied. The cell membrane can undergo a rearrangement in their morphology after exceeding a critical threshold. Therefore, pores can be formed on the membrane. The pores can be large enough to transport molecules and ions across the membrane. Different types of electroporation can result based on the intensity and duration of the applied electric field. Reversible electroporation (RE) can occur when the electric field is high enough to exceed the critical threshold, but the cell membrane can be still able to return to its initial state. Irreversible electroporation (IRE) can occur when the electric field is extremely high, and the number of pores created induce osmotic imbalance or homeostasis loss in the cell, resulting in cell death. Thus, electroporation can be used to increase the permeability of the cell membrane, allowing membrane-impermeable substance (such as peptides, antibodies, DNA molecules, RNA molecules, chemicals, dugs) to be introduced into the cell. The present method can be used to perform electroporation on a single cell. In some embodiments, the electroporation is reversible. In some embodiments, the electroporation is irreversible. For example, upon application of the electric field to the plurality of microwells, electroporation can occur in the single cell in the microwell, thereby the reaction reagent introduced in the microwell can enter the cell.

The interaction between the between the reaction reagent and the biomolecule can include, but is not limited to, include antibody-antigen binding, nucleic acids hybridization, ligand-protein complexing, ligand-receptor binding, protein-protein binding, chemical complexing, or a combination thereof. The detection of the biomolecule can include, for example, spectrometric analysis (e.g., mass spectrometry), fluorescence analysis (e.g., fluorescence spectroscopy), sequencing (e.g., DNA or RNA sequencing), and combinations thereof.

The reaction reagent entering the cell can be a probe capable of capturing the biomolecule. As used herein, capturing of the biomolecule by a probe can include any interaction between the probe and the biomolecule that results in the labeling, tagging, indexing, immobilizing, or otherwise characterizing the biomolecule. For example, the probe can be hybridized to, bind to, or form a covalent bond with, the biomolecule. The captured biomolecule can be identified or isolated from other biomolecules that are not captured by the probe. In some embodiments, the probe is a small molecule compound, a polypeptide, an oligonucleotide, a ribonucleic acid (RNA), a deoxyribonucleic acid (DNA), an oligosaccharide, a sugar, or a combination thereof.

For example, the probe can be a barcode molecule as disclosed herein, and the biomolecule can be a nucleic acid associate with the cell. The probe (barcode molecule) can be introduced, for example, by partitioning a plurality of particles each comprising a plurality of probes (barcode molecules) into the microwell as describe herein. The interaction of the probe and the biomolecule can include a barcoding reaction of the nucleic acid using the barcode molecule to generate a barcoded nucleic acid. Detection of the biomolecule can include analyzing the barcoded nucleic acid, such as determining the sequence of the barcode nucleic acid.

Disclosed herein also include methods of analyzing a biomolecule. The method can include, within a partition comprising a biomolecule, applying an electric field, thereby manipulating a movement of the biomolecule within the partition. The method can further include analyzing the biomolecule.

The biomolecule as disclosed herein can have one or more positively charged groups (such as ammonium group, —NH₃⁺), one or more negatively charged groups (such as carboxylate group, —COO⁻, or phosphate group), or both. The biomolecule can be positively charged or negatively charged. In some embodiments, the biomolecule is a polypeptide, a protein, an oligonucleotide, a ribonucleic acid (RNA), a deoxyribonucleic acid (DNA), an oligosaccharide, a sugar, or a combination thereof. For example, the biomolecule can be a target nucleic acid as disclosed herein. The nucleic acid can be negatively charged.

In some embodiments, the biomolecule is associated or previously associated with a single cell. For example, the biomolecule (e.g., a nucleic acid) can be a component of the single cell, and can be released from the single cell (e.g., by cell lysis). In some embodiments, the methods of analyzing a biomolecule can include introducing (e.g., partitioning) the single cell into the partition. In some embodiments, the method also include releasing the biomolecule from the single cell.

The biomolecule (e.g., a nucleic acid) can be analyzed, for example, by barcoding and/or sequencing. In some embodiments, the methods of analyzing a biomolecule (e.g., a nucleic acid associated with a single cell) further includes introducing (e.g., partitioning) a plurality of barcode molecules into the partition. For example, the barcode molecules can be introduced by partitioning a plurality of particles each comprising a plurality of barcode molecules into the partition as describe herein.

The barcode molecule can be used, for example, to barcode the biomolecules to generate barcoded biomolecule (e.g. barcoded nucleic acids). The method can further include barcoding the biomolecule using the plurality of barcode molecules. For example, the method can further include barcoding target nucleic acids associated with the single cell in the partition using the plurality of barcode molecules in the partition Analyzing the biomolecule can include, for example, analyzing the barcoded biomolecule (such as barcoded nucleic acids).

### Device and Electric Field

In some embodiments, the present methods can be carried out using the microfluidic devices as described herein. For example, the cells and particles can be introduced into microwells of the microwell array of the present microfluidic device. The electric field used in the present methods can be applied, for example, to the first and second electrically conductive layers of the present microfluidic device. As a result, an electrophoresis can occur in the microwells of the present microfluidic device, and a biomolecule (such as a target nucleic acid associated with a single cell can be manipulated and analyzed according to the present methods.

An electric potential (or voltage) of the electric field can be, be about, be at least, be at least about, be at most, or be at most about, 0.001 V, 0.01 V. 0.1 V, 0.2 V, 0.3 V, 0.4 V, 0.5 V, 0.6 V, 0.7 V. 0.8 V, 0.9 V, 1 V. 2 V, 3 V, 4 V, 5 V, 6 V, 7 V, 8 V, 9 V, 10 V, 20 V, 30 V, 40 V, 50 V, 60 V, 70V, 80 V, 90 V, 100 V, 200 V, 300 V, 400 V, 500 V, 600 V, 700 V, 800 V, 900 V, 1000 V, 10000 V, 100000V, or a number or a range between any two of these values. In some embodiments, the voltage of the electric field is about 0.1 V to 10 V.

The electric field can be used to control the movement of the an electronically charged biomolecule associated with the cell. The biomolecule (e.g., a negatively charged nucleic acid) can be released from the cell before, while, or after the electric field is applied. In some embodiments, applying the electric field to the microwell is performed before releasing the plurality of target nucleic acids associated with the single cell in the microwell of the plurality of microwells. In some embodiments, releasing the plurality of target nucleic acids includes: while applying the electric field to the microwell array, releasing the plurality of target nucleic acids associated with the single cell in the microwell of the plurality of microwells. In some embodiments, applying the electric field to the microwell is performed after releasing the plurality of target nucleic acids associated with the single cell in the microwell of the plurality of microwells.

The electric field can be used to facilitate a reaction in the present methods. In some embodiments, the methods disclosed herein further include, while applying the electric field, introducing a first reaction reagent into the microwell and performing a reaction on a content of the single cell in the microwell using the first reaction reagent. The methods can further include, while applying the electric field, introducing a second reaction reagent into the microwell and performing a second reaction on a content of the single cell in the microwell using the second reaction reagent. For example, the content of the single cell can include cell membrane, nuclear membrane, biomolecules associated with the cell, or combinations thereof. The first reaction reagent can include a lysis buffer, and the first reaction can include a lysis reaction of the cell. The second reaction reagent can include a barcode molecule or a probe capable of capturing a biomolecule (e.g., a nucleic acid) associated with the cell, and the second reaction can include barcoding the biomolecule using the barcode molecule. As another example, the first reaction can include a reaction of a first biomolecule, and the second reaction can include a reaction of a second biomolecule, which is different from the first biomolecule.

The first reaction reagent and the second reaction reagent can be same or different. In some embodiments, the first reaction reagent and the second reaction reagent are different. Different reaction reagents can be introduced sequentially into the microwell while applying the electric field. For example, the first reaction reagent can be removed after the first reaction, and the second reaction reagent can then be introduced to perform the second reaction. The biomolecules (e.g., negatively charged nucleic acids) in this process can be restricted or immobilized in the microwell, which improves the mixing efficiency of the biomolecule with the first and second reaction reagents, thus improving the efficiency of the first and second reaction. Accordingly, the microfluidic device and methods disclosed herein can be used to perform reaction exchange reactions.

In some embodiments, the electric field can restrict the movement the plurality of target nucleic acids or biomolecule in the partition (e.g., a microwell). For example, as a result of the restriction by the electric field, a target nucleic acid of the cell or a biomolecule can only move within the partition, move in one direction in the partition, or be immobilized in the partition (e.g., at the bottom of the microwell). In some embodiments, the partition (e.g., a microwell) can include an open end and a closed end facing the open end (e.g., bottom of the microwell). The electric field can be applied, for example, in a direction from the opened end to the closed end, or an opposite direction thereof. When the electric field is applied, a biomolecule (e.g., a negatively charged nucleic acid) can move in the direction of the electric field, or opposite thereof, in the partition. For example, the biomolecule can move toward the closed end of the partition

### Partition

A partition as used herein refers to a part, a portion, or a division sequestered from the rest of the parts, portions, or divisions. A partition can be formed through the use of wells, microwells, multi-well plates, microwell arrays, microfluidics, dilution, dispensing, droplets, or any other means of sequestering one fraction of a sample from another. In some embodiments, the partition is a droplet or a microwell of a microwell array comprising a plurality of microwells. For example, the partition can be a microwell of a microwell array comprising a plurality of microwells.

The plurality of partitions can include at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, or 500000 partitions. In some embodiments, the plurality of partitions includes at least 100 partitions.

### Microwell Array

In some embodiments, the plurality of partitions include a plurality of microwells of a microwell array. The microwell array can include different numbers of microwells in different implementations. In some embodiments, the microwell array can include, include about, include at least, include at least about, include at most, or include at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values, microwells. The microwells can be arranged into rows and columns, for example. The number of microwells in a row (or a column) can be, be about, be at least, be at least about, be at most, or be at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, or a number or a range between any two of these values. Adjacent rows (or columns) of microwells can be aligned or staggered, for example.

The width, length, depth (or height), radius, or diameter of a microwell of the plurality of microwells can be different in different implementations. In some embodiments, the width, length, depth (or height), radius, or diameter of a microwell of the plurality of microwells can be, be about, be at least, be at least about, be at most, or be at most about, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 600 μm, 700 μm, 800 μm, 900 μm, 1000 μm, or a number or a range between any two of these values. For example, the width of a microwell of the plurality of microwells is 10 μm to 200 μm. As another example, the length of a microwell of the plurality of microwells can be 10 μm to 200 μm. As a further example, the depth of a microwell of the plurality of microwells can be 5 μm to 500 μm. In the non-limiting exemplary embodiment, the width of a microwell is 10 μm, the length of a microwell is 20 μm to 100 μm, such as 20 μm, and the depth of a microwell is 5 μm to 10 μm. The shape of a microwell can be different in different embodiments. In some embodiments, a microwell has a circular, elliptical, square, rectangular, triangular, or hexagonal shape.

The volume of one, one or more, or each, of the plurality of microwells can be different in different embodiments. The volume of one, one or more, or each, of the plurality of microwells can be, be about, be at least, be at least about, be at most, or be at most about, 1 nm³, 2 nm³, 3 nm³, 4 nm³, 5 nm³, 6 nm³, 7 nm², 8 nm³, 9 nm³, 10 nm³, 20 nm², 30 nm³, 40 nm³, 50 nm³, 60 nm³, 70 nm³, 80 nm³, 90 nm³, 100 nm³, 200 nm³, 300 nm³, 400 nm³, 500 nm³, 600 nm³, 700 nm³, 800 nm³, 900 μm³, 1000 nm³, 10000 nm³, 100000 μm³, 1000000 nm³, 10000000 nm³. 100000000 μm³, 1000000000 nm³, 2 μm³, 3 μm³, 4 μm³, 5 μm³, 6 μm³, 7 μm³, 8 μm³, 9 μm³, 10 μm³, 20 μm³, 30 μm³, 40 μm³, 50 μm³, 60 μm³, 70 μm³, 80 μm³, 90 μm³, 100 μm³, 200 μm³, 300 μm³, 400 μm³, 500 μm³, 600 μm³, 700 μm³, 800 μm³, 900 μm³, 1000 μm³, 10000 μm³, 100000 μm³, 1000000 μm³, or a number or a range between any two of these values. The volume of one, one or more, or each, of the plurality of microwells can be, be about, be at least, be at least about, be at most, or be at most about, 1 nanolieter (nl), 2 nl, 3 nl, 4 nl, 5 nl, 6 nl, 7 nl, 8 nl, 9 nl, 10 nl, 11 nl, 12 nl, 13 nl, 14 nl, 15 nl, 16 nl, 17 nl, 18 nl, 19 nl, 20 nl, 21 nl, 22 nl, 23 nl, 24 nl, 25 nl, 26 nl, 27 nl, 28 nl, 29 nl, 30 nl, 31 nl, 32 nl, 33 nl, 34 nl, 35 nl, 36 nl, 37 nl, 38 nl, 39 nl, 40 nl, 41 nl, 42 nl, 43 nl, 44 nl, 45 nl, 46 nl, 47 nl, 48 nl, 49 nl, 50 nl, 51 nl, 52 nl, 53 nl, 54 nl, 55 nl, 56 nl, 57 nl, 58 nl, 59 nl, 60 nl, 61 nl, 62 nl, 63 nl, 64 nl, 65 nl, 66 nl, 67 nl, 68 nl, 69 nl, 70 nl, 71 nl, 72 nl, 73 nl, 74 nl, 75 nl, 76 nl, 77 nl, 78 nl, 79 nl, 80 nl, 81 nl, 82 nl, 83 nl, 84 nl, 85 nl, 86 nl, 87 nl, 88 nl, 89 nl, 90 nl, 91 nl, 92 nl, 93 nl, 94 nl, 95 nl, 96 nl, 97 nl, 98 nl, 99 nl, 100 nl, or a number or a range between any two of these values. For example, the volume of one, one or more, or each, of the plurality of microwells is about 1 nm² to about 1000000 μm².

The microwell array comprising a plurality of microwells can be formed from any suitable material as will be understood by a person of skill in the art. In some embodiments, a microwell array comprising a plurality of microwells can be formed from a material selected from the group consisting of silicon, glass, ceramic, elastomers such as polydimethylsiloxane (PDMS) and thermoset polyester, thermoplastic polymers such as polystyrene, polycarbonate, poly(methyl methacrylate) (PMMA), poly-ethylene glycol diacrylate (PEGDA), Teflon, polyurethane (PU), composite materials such as cyclic-olefin copolymer, and combinations thereof.

Partitions (e.g., microwells) described above can be introduced with samples, free reagents, and/or reagents encapsulated in microcapsules. The reagents can include restriction enzymes, ligase, polymerase, fluorophores, oligonucleotide barcodes, oligonucleotide probes, adapters, buffers, dNTPs, ddNTPs, and other reagents required for performing the methods described herein.

### Partitioning Cells

In some embodiments, the cells are partitioned into a plurality of microwells of a microwell array. As a result of partitioning, the percentage of the plurality of microwells of the microwell array each comprising a single cell of the plurality of cells can vary. For example, the percentage of the plurality of microwells of the microwell array each comprising a single cell can be, be about, be at least, be at least about, be at most, or be at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, at least 25% of the plurality of microwells can each include a single cell of the plurality of cells.

As a result of partitioning, the percentage of the plurality of microwells of the microwell array each comprising at most one of the plurality of cells can vary. For example, the percentage of the plurality of microwells of the microwell array each comprising at most one of the plurality of cells can be, be about, be at least, be at least about, be at most, or be at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, as a result of the partitioning, at least 90% of the plurality of microwells can each include at most one of the plurality of cells.

The percentage of the plurality of partitions comprising no cell can be different in different embodiments. For example, the percentage of the plurality of partitions comprising no cell can be, be about, be at least, be at least about, be at most, or be at most about, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. In some embodiments, at most 50% of partitions of the plurality of partitions can include no cell of the plurality of cells.

The percentage of the plurality of partitions comprising two or more cells can be different in different embodiments. For example, the percentage of the plurality of partitions comprising two or more cells can be, be about, be at least, be at least about, be at most, or be at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. In some embodiments, at most 10% of partitions of the plurality of partitions can include two or more of the plurality of cells.

As a result of co-partitioning a plurality of cells and a plurality of particles into a plurality of microwells of a microwell array, the percentage of the plurality of microwells of the microwell array each comprising a single cell of the plurality of cells and a single particle of the plurality of particles can vary. For example, the percentage of the plurality of microwells of the microwell array each comprising a single cell and a single particle can be, be about, be at least, be at least about, be at most, or be at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, at least 25% of the plurality of microwells can each include a single cell of the plurality of cells and a single particle of the plurality of particles.

### Cells

The cells can be obtained from any organism of interest. A cell can be a mammalian cell, and particularly a human cell such as T cells, B cells, natural killer cells, stem cells, cancer cells.

Cells described herein can be obtained from, derived from, cultured from, or progenies of cells cultured from a cell sample. A cell sample comprising cells can be obtained from any source including a clinical sample and a derivative thereof, a biological sample and a derivative thereof, a forensic sample and a derivative thereof, and a combination thereof. A cell sample can be collected from any bodily fluids including, but not limited to, blood, urine, serum, lymph, saliva, anal, and vaginal secretions, perspiration and semen of any organism. A cell sample can be products of experimental manipulation including purification, cell culturation, cell isolation, cell separation, cell quantification, sample dilution, or any other cell sample processing approaches. A cell sample can be obtained by dissociation of any biopsy tissues of any organism including, but not limited to, skin, bone, hair, brain, liver, heart, kidney, spleen, pancreas, stomach, intestine, bladder, lung, esophagus.

In some embodiments, the cell sample is a clinical sample or a derivative thereof, a biological sample or a derivative thereof, an environmental sample or a derivative thereof, a forensic sample or a derivative thereof, or a combination thereof. In some embodiments, the cell sample is collected from blood, urine, serum, lymph, saliva, anal, and vaginal secretions, perspiration, and/or semen of any organism. In some embodiments, the cell sample is obtained from skin, bone, hair, brain, liver, heart, kidney, spleen, pancreas, stomach, intestine, bladder, lung, and/or esophagus of any organism. In some embodiments, the cells are cultured cells, such as cells from a cultured cell line. In some embodiments, the cells include immune cells, fibroblast cells, stem cells, or cancer cells.

### Target Nucleic Acids

As described herein, cells can be associated with target nucleic acids. For example, a cell can include one or more target nucleic acids (e.g., mRNA) or can be labeled with one or more target nucleic acids (e.g., directly, or indirectly through a binding moiety, such as an antibody conjugated with the nucleic acid). The target nucleic acids associated with the cell can be from, on the surface of, or binding to the surface of the cell. A target nucleic acid can have a sequence (e.g., an mRNA sequence, excluding the poly(A) tail).

The target nucleic acids associated with the cell can include deoxyribonucleic acid (DNA), ribonucleic acid (RNA), and/or any combination or hybrid thereof. The target nucleic acids can be single-stranded or double-stranded, or contain portions of both double-stranded or single-stranded sequences. The target nucleic acids can contain any combination of nucleotides, including uracil, adenine, thymine, cytosine, guanine, inosine, xanthine, hypoxanthine, isocytosine, isoguanine and any nucleotide derivative thereof. As used herein, the term “nucleotide” can include naturally occurring nucleotides and nucleotide analogs, including both synthetic and naturally occurring species. The target nucleic acids can be genomic DNA (gDNA), mitochondrial DNA (mtDNA), messenger RNA (mRNA), ribosomal RNA (rRNA), transfer RNA (tRNA), nuclear RNA (nRNA), small interfering RNA (siRNA), small nuclear RNA (snRNA), small nucleolar RNA (snoRNA), small Cajal body-specific RNA (scaRNA), microRNA (miRNA), double stranded (dsRNA), ribozyme, riboswitch or viral RNA, or any nucleic acids that may be obtained from a sample.

The plurality of target nucleic acids can, for example, include DNA, genomic DNA (gDNA), ribonucleic acid (RNA), and/or messenger RNA (mRNA). In some embodiments, the plurality of target nucleic acids includes mRNA, for example a poly-adenylated mRNA.

### Barcoding

The barcode molecules introduced into the partitions (e.g., microwells or droplets) can be associated with particles (e.g., beads). A plurality of particles can be introduced (e.g., partitioned) into the partitions. In some embodiments, each of the plurality of particles includes a plurality of barcode molecules. The particles can provide a surface upon which molecules, such as oligonucleotides, can be synthesized or attached. In some embodiments, the plurality of barcode molecules are attached to, reversibly attached to, covalently attached to, or irreversibly attached to the particle. In some embodiments, the particle is a bead.

The particle can include, include about, include at least, include at least about, include at most, or include at most about, 10, 50, 100, 1000, 10000, 20000, 30000, 40000, 50000, 60000, 70000, 80000, 90000, 100000, 200,000, 300,000, 400,000, 500,000, 600,000, 700,000, 800,000, 900,000, 1000000, 2000000, 3000000, 4000000, 5000000, 6000000, 7000000, 8000000, 9000000, 10000000, or a number or a range between any two of these values, barcode molecules. The attachment of barcode molecules to the particle can be covalent or non-covalent via non-covalent bonds such as ionic bonds, hydrogen bonds, or van der Waals interactions. The attachment can be direct to the surface of a particle or indirect through other oligonucleotide sequences attached to the surface of a particle.

The particle (e.g., a bead) can be dissolvable, degradable, or disruptable. A particle can be a gel particle such as a hydrogel particle. In some embodiments, the gel particle is degradable upon application of a stimulus. The stimulus can include a thermal stimulus, a chemical stimulus, a biological stimulus, a photo-stimulus, or a combination thereof. The particle can be a solid particle and/or a magnetic particle. In some embodiments, the particle is a magnetic particle. The magnetic particle can include a paramagnetic material coated or embedded in the magnetic particle (e.g. on a surface, in an intermediate layer, and/or mixed with other materials of the magnetic particle). A paramagnetic material refers to a material having a magnetic susceptibility slightly greater than 1 (e.g. between about 1 and about 5). A magnetic susceptibility is a measure of how much a material can become magnetized in an applied magnetic field. Paramagnetic materials include, but not limited to, magnesium, molybdenum, lithium, aluminum, nickel, tantalum, titanium, iron oxide, gold, copper, or a combination thereof. In some embodiments, the magnetic particle comprising barcode molecules can be immobilized or retained in a partition (such as a microwell) by an external magnetic field, thereby retaining the barcode molecules in a partition. The magnetic particle comprising barcode molecules can be mobilized or released when the external magnetic field is removed.

In some embodiments, a particle can be immobilized or retained in a partition (e.g., a microwell) through an interaction between two members of a binding pair. For example, the partition (e.g., microwell) can be coated with a capture moiety (e.g., a member of a binding pair) capable of binding with a binding moiety (the other member of the binding pair) included in or conjugated to a particle, such that the binding of the two moieties results in the attachment of the particle to the partition (e.g., microwell), thereby immobilizing or retaining the particle in the partition. For example, the surface of a partition (e.g, microwell) can be coated with streptavidin. The biotinylated particle can be attached to the surface of the partition (e.g., microwell) via streptavidin-biotin interaction.

Particles can be of uniform size or heterogeneous size. In some embodiments, a particle can be sized such that at most one particle, not two particles, can fit one partition. A size or dimension (e.g., length, width, depth, radius, or diameter) of a particle can be different in different embodiments. For example, a size or dimension of one, or each, particle can be, be about, be at least, be at least about, be at most, or be at most about, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, or a number or a range between any two of these values. In some embodiments, a size or dimension of one, or each, particle is about 1 nm to about 100 μm. In some embodiments, the particle can have a size of about 10 μm to about 100 μm. In some embodiments, the particle can have a size of about 30 μm.

The volume of one, or each, particle can vary. The volume of one, or each, particle can be, be about, be at least, be at least about, be at most, or be at most about, 1 nm³, 2 nm³, 3 nm³, 4 nm³, 5 nm³, 6 nm³, 7 nm³, 8 nm³, 9 nm³, 10 nm³, 20 nm³, 30 nm³, 40 nm³, 50 nm³, 60 nm³, 70 nm³. 80 nm³, 90 nm³, 100 nm³, 200 nm³, 300 nm³, 400 nm³, 500 nm³, 600 nm³, 700 nm³, 800 nm³, 900 μm³, 1000 nm³, 10000 nm³, 100000 μm³, 1000000 nm³, 10000000 nm³, 100000000 μm³, 1000000000 nm³, 2 μm, 3 μm, 4 μm, 5 μm, 6 μm, 7 μm, 8 μm, 9 μm, 10 μm, 20 μm³, 30 μm³, 40 μm³, 50 μm³, 60 μm³, 70 μm³, 80 μm³, 90 μm³, 100 μm³, 200 μm³, 300 μm², 400 μm³, 500 μm³, 600 μm³, 700 μm², 800 μm³, 900 μm³, 1000 μm³, 10000 μm², 100000 μm³, 1000000 μm³, or a number or a range between any two of these values. The volume of one, or each, particle can be, be about, be at least, be at least about, be at most, or be at most about, 1 nanolieter (nL), 2 nL, 3 nL, 4 nL, 5 nL, 6 nL, 7 nL, 8 nL, 9 nL, 10 nL, 11 nL, 12 nL, 13 nL, 14 nL, 15 nL, 16 nL, 17 nL, 18 nL, 19 nL, 20 nL, 21 nL, 22 nL, 23 nL, 24 nL, 25 nL, 26 nL, 27 nL, 28 nL, 29 nL, 30 nL, 31 nL, 32 nL, 33 nL, 34 nL, 35 nL, 36 nL, 37 nL, 38 nL, 39 nL, 40 nL, 41 nL, 42 nL, 43 nL, 44 nL, 45 nL, 46 nL, 47 nL, 48 nL, 49 nL, 50 nL, 51 nL, 52 nL, 53 nL, 54 nL, 55 nL, 56 nL, 57 nL, 58 nL, 59 nL, 60 nL, 61 nL, 62 nL, 63 nL, 64 nL, 65 nL, 66 nL, 67 nL, 68 nL, 69 nL, 70 nL, 71 nL, 72 nL, 73 nL, 74 nL, 75 nL, 76 nL, 77 nL, 78 nL, 79 nL, 80 nL, 81 nL, 82 nL, 83 nL, 84 nL, 85 nL, 86 nL, 87 nL, 88 nL, 89 nL, 90 nL, 91 nL, 92 nL, 93 nL, 94 nL, 95 nL, 96 nL, 97 nL, 98 nL., 99 nL, 100 nL, or a number or a range between any two of these values. In some embodiments, the volume of one, or each, particle is about 1 nm³ to about 1000000 μm³.

The number of particles introduced into a plurality of partitions can be different in different embodiments. In some embodiments, the number of particles introduced into a plurality of partitions is, is about, is at least, is at least about, is at most, or is at most, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, or a number or a range between any two of these values.

In some embodiments, particles are introduced to the partitions such that the percentage of partitions each occupied with a single particle is, is about, is at least, is at least about, is at most, or is at most about, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, at least 25% of the plurality of partitions is each occupied with a single particle.

In some embodiments, particles are introduced to the partitions such that the percentage of partitions with no particle is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values. In some embodiments, at most 20% of the plurality of partitions contain no particle.

In some embodiments, analyzing the plurality of target nucleic acids associated with the cell can includes barcoding the plurality of target nucleic acids using the plurality of barcode molecules to generate a plurality of barcoded nucleic acids; and analyzing the plurality of barcoded nucleic acids, or products thereof. In some embodiments, analyzing the plurality of barcoded target nucleic acids, or products thereof, includes determining the sequences of the plurality of barcoded target nucleic acids, or products thereof.

In some embodiments, analyzing the plurality of barcoded nucleic acids includes analyzing the sequences of the barcoded nucleic acids. In some embodiments, analyzing the sequences of the barcoded nucleic acids includes: determining a profile of one or more cells from the sequences of the barcoded nucleic acids.

### Barcode Molecules

Barcode molecules (e.g., barcode molecules attached to particles) can be partitioned, for example, in microwells. The term “barcode” as used herein can be a verb or a noun. When used as a noun, the term “barcode” or “barcode molecule” refers to a label that can be attached to a polynucleotide, or any variant thereof, to convey information about the polynucleotide. For example, a barcode can be a polynucleotide sequence attached to fragments of the target nucleic acids associated with a cell in the partition. The barcode can then be sequenced alone or with the fragments of the target nucleic acids associated with the cell. The presence of the same barcode on multiple sequences or different barcodes on different sequences can provide information about the cell origin and/or the molecular origin of the sequences. When used as a verb, the term “barcode” refers to a process of attaching a barcode or a barcode molecule to a target nucleic acid associated with the cell.

Barcode molecules can be generated from a variety of different formats, including pre-designed polynucleotide barcodes, randomly synthesized barcode sequences, microarray-based barcode synthesis, random N-mers, or combinations thereof as will be understood by a person skilled in the art.

In some embodiments, the plurality of barcode molecules include, include about, include at least, include at least about, include at most, or include at most about 1, 5, 10, 50, 100, 1000, 10000, 20000, 30000, 40000, 50000, 60000, 70000, 80000, 90000, 100000, 200,000, 300,000, 400,000, 500,000, 600,000, 700,000, 800,000, 900,000, 1000000, 2000000, 3000000, 4000000, 5000000, 6000000, 7000000, 8000000, 9000000, 10000000, 20000000, 50000000, 100000000, 200000000, 300000000, 400000000, 500000000, 600000000, 700000000, 800000000, 900000000, 1000000000, or a number or a range between any two of these values.

A barcode molecule (or a segment of a barcode molecule, such as a particle barcode sequence or a molecular barcode sequence) can be in any suitable length. For example, a barcode molecule (or a segment of a barcode molecule) can be about 2 to about 500 nucleotides in length, about 2 to about 100 nucleotides in length, about 2 to about 50 nucleotides in length, about 2 to about 40 nucleotides in length, about 4 to about 20 nucleotides in length, or about 6 to 16 nucleotides in length. In some embodiments, the barcode molecule (or a segment of a barcode molecule) can be, be about, be at least, be at least about, be at most, or be at most about 2, 3, 4, 5, 6, 7, 8, 9, 10, 11, 12, 13, 14, 15, 16, 17, 18, 19, 20, 25, 30, 35, 40, 45, 50, 55, 60, 65, 70, 85, 90, 95, 100, 150, 200, 250, 300, 400, or 500 nucleotides in length, or a number or a range between any two of these values.

The barcode molecules used herein can include a particle barcode sequence and a molecular barcode sequence (e.g. a unique molecular identifier (UMI)). A barcode molecule can also include additional sequences, such as a target binding sequence or region capable of hybridizing to target nucleic acids (e.g. poly(dT) sequence), other recognition or binding sequences, a template switching oligonucleotide (e.g., GGG, such as rGrGrG), and primer sequences (e.g. sequencing primer sequence, such as Read 1 or a PCR primer sequence) for subsequent processing (e.g. PCR amplification) and/or sequencing.

The configuration of the various sequences included in a barcode molecule (e.g. particle barcode sequence, UMI, primer sequence, target binding sequence or region, and/or any additional sequences) can vary depending on, for example, the particular configuration desired and/or the order in which the various components of the sequence are added as will be understood to a person skilled in the art. In some embodiments, a barcode molecule has a configuration of 5′-primer sequence-particle barcode sequence-UMI-target binding sequence-3′. In some embodiments, a barcode molecule has a configuration of 5′-primer sequence-particle barcode sequence-UMI-template switching oligonucleotide-3′.

### Particle Barcode Sequence

In some embodiments, the barcode molecules can include a particle barcode sequence. Particle barcode sequences can be used to identify the barcoded nucleic acids originate from the cell. (or the same partition). Barcoded nucleic acids that originate from the cell (or the same partition) can have an identical particle barcode sequence. A particle barcode sequence can be referred to as a partition specific barcode, such as a microwell specific barcode, or a sample barcode. The particle barcode sequence of the barcode molecules in a partition can be identical or different.

In some embodiments, the particle barcode sequences can serve to track the target nucleic acids associated with the cell throughout the processing (e.g., location of the cells in a plurality of partitions, such as microwells) when the particle barcode sequence associated with the target nucleic acids is determined during sequencing.

The number (or percentage) of barcode molecules introduced in a partition with particle barcode sequences having an identical sequence can be different in different embodiments. In some embodiments, the number of barcode molecules introduced in a partition with particle barcode sequences having an identical sequence is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of barcode molecules introduced in a partition with particle barcode sequences having an identical sequence is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values. For example, the particle barcode sequences of at least two barcode molecules introduced in a partition include an identical sequence. In some embodiments, at least two of the particle barcode sequences of the plurality of barcode molecules in the same partition are identical.

The percentage of barcode molecules on a single particle with particle barcode sequences having an identical sequence is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values. For example, the particle barcode sequences of the plurality of barcode molecules on a single particle are identical.

A particle barcode sequence can be unique (or substantially unique) to a partition. The number of unique particle barcode sequences can be different in different embodiments. In some embodiments, the number of unique particle barcode sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of unique particle barcode sequences is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values, of the particle barcode sequences of the barcode molecules introduced in a partition. For example, the particle barcode sequences of barcode molecules introduced in two partitions can include different sequences. In some embodiments, the particle barcode sequences of at least one barcode molecules in at least two different partitions are different.

In some embodiments, barcode molecules are introduced to the plurality of partitions such that different sets of a plurality of barcode molecules introduced in different partitions have different particle barcode sequences and a same set of plurality of barcode molecules introduced in a same partition have same particle barcode sequence. For example, target nucleic acids associated with a cell in a partition (e.g., a microwell) can be barcoded with the same particle barcode sequences.

The length of a particle barcode sequence of a barcode molecule (or a particle barcode sequence of each barcode molecule or all particle barcode sequences of the plurality of barcode molecules) can be different in different embodiments. In some embodiments, a particle barcode sequence of a barcode molecule (or each particle barcode sequence of each barcode molecule or all particle barcode sequences of the plurality of barcode molecules) is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

### Molecular Barcode Sequence

In some embodiments, a barcode molecular can include a molecular barcode sequence. Molecular barcode sequences can be unique molecule identifiers (UMIs). Molecular barcode sequences can be used to identify molecular origins of the barcoded nucleic acids. Molecular barcode sequences (e.g., UMIs) are short sequences used to uniquely tag each molecule in a sample in some embodiments. The molecular barcode sequences of the barcode molecules partitioned into a partition can be identical or different.

In some embodiments, the molecular barcode sequences of the plurality of barcode molecules are different. The number (or percentage) of molecular barcode sequences of barcode molecules introduced in a partition (e.g., a microwell) with different sequences can be different in different embodiments. In some embodiments, the number of molecular barcode sequences of barcode molecules introduced in a partition with different sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of molecular barcode sequences of barcode molecules introduced in a partition with different sequences is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values. For example, the molecular barcode sequences of two barcode molecules of the plurality of barcode molecules introduced in a partition can include different sequences.

The number of barcode molecules introduced in a partition with molecular barcode sequences having an identical sequence can be different in different embodiments. In some embodiments, the number of barcode molecules introduced in a partition with molecular barcode sequences having an identical sequence is, is about, is at least, is at least about, is at most, or is at most about, 1, 2, 3, 4, 5, 6, 7, 8, 9, 10, 20, 30, 40, 50, 60, 70, 80, 90, 100, or a number or a range between any two of these values. For example, the molecular barcode sequences of two barcode molecules introduced in a partition can include an identical sequence.

The number of unique molecular barcode sequences can vary. For example, the number of unique molecular barcode sequences can be, be about, be at least, be at least about, be at most, or be at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values.

In some embodiments, at least two of the molecular barcode sequences of the plurality of barcode molecules in a partition include different molecular barcode sequences (e.g., unique molecular identifiers).

The length of a molecular barcode sequence of a barcode molecule (or a molecular barcode sequence of each barcode molecule) can be different in different embodiments. In some embodiments, a molecular barcode sequence of a barcode molecule (or a molecular barcode sequence of each barcode molecule) is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. In some embodiments, the molecular barcode sequences (e.g., UMIs) can be 2-40 nucleotides in length.

In some embodiments, a barcode molecule (or each of the plurality of barcode molecules) can include a primer sequence. The primer sequence can be a sequencing primer sequence (or a sequencing primer binding sequence) or a PCR primer sequence (or PCR primer binding sequence). For example, the sequencing primer can be a Read 1 sequence, a Read 2 sequence, or a portion thereof. In some embodiments, the barcode molecule include a PCR primer binding sequence, which allows for PCR amplification of a barcoded nucleic acid.

The length of the primer sequence can vary. In some embodiments, the primer sequence is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. The number (or percentage) of barcode molecules in a partition (eg., a microwell) each comprising a primer sequence (or each comprising an identical primer sequence) can be different in different embodiments. In some embodiments, the number of barcode molecules in a partition (e.g., a microwell) each comprising a primer sequence (such as a PCR primer binding sequence) is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. In some embodiments, the percentage of barcode molecules in a partition (e.g., a microwell) each comprising a primer sequence (or each comprising an identical primer sequence) is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

In some embodiments, each of the plurality of barcode molecules includes a primer sequence (e.g., a sequencing primer sequence, including but not limited to, a Read 1 sequence, a Read 2 sequence, or a portion thereof).

In some embodiments, a barcode molecule includes a target binding sequence or region capable of hybridizing to the target nucleic acids, a particular type of target nucleic acids (e.g. mRNA), and/or specific target nucleic acids (e.g. specific gene of interest). In some embodiments, the target binding sequence includes a poly(dT) sequence and/or a sequence capable of hybridizing to the plurality of target nucleic acids.

The length of a target binding sequence can vary. For example, the target binding sequence can be, be about, be at least, be at least about, be at most, or be at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. The target binding sequence can be 12-18 deoxythymidines in length. In some embodiments, the target binding sequence can be 20 nucleotides or longer to enable their annealing in reverse transcription reactions at higher temperatures as will be understood by a person of skill in the art.

In some embodiments, barcode molecules comprising target binding sequences are introduced into the partitions together with other reagents such as the reverse transcription reagents. The number of the barcode molecules introduced into a partition comprising a target binding sequence can vary. For example, the number of barcode molecules introduced into a partition comprising a target binding sequence (e.g., poly(dT) sequence) can be, be about, be at least, be at least about, be at most, or be at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, or a number or a range between any two of these values.

In some embodiments, the target binding sequence can be on a 3′ end of a barcode molecule of the plurality of barcode molecules introduced in a partition. Barcode molecules each comprising a poly(dT) target binding sequence can be used to capture (e.g., hybridize to) 3′ end of polyadenylated mRNA transcripts in a target nucleic acid for a downstream 3′ gene expression library construction.

In some embodiments, the target binding sequence can include a poly(dT) sequence which is a single-stranded sequence of deoxythymidine (dT) used for first-strand cDNA synthesis catalyzed by reverse transcriptase. In some embodiments, the target binding sequence includes a poly(dT) sequence can be introduced into the partitions as extension primers to synthesize the first-strand cDNA using the target nucleic acid (e.g. RNA) as a template.

In some embodiments, the poly(dT) of the barcode molecules introduced into a partition are identical (e.g., same number of dTs). In some embodiments, the poly(dT) of the barcode molecules introduced into a partition are different (e.g. different numbers of dTs). The percentage of the barcode molecules of the plurality of barcode molecules introduced into a partition with an identical poly(dT) sequence can vary. In some embodiments, the percentage of the barcode molecules of the plurality of barcode molecules introduced into a partition with an identical poly(dT) sequence is, is about, is at least, is at least about, is at most, is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

In some embodiments, the target binding regions of all barcode molecules of the plurality of barcode molecules include poly(dT) capable of hybridizing to poly(A) tails of mRNA molecules (or poly(dA) regions or tails of DNA). In some embodiments, the target binding regions of some barcode molecules of the plurality of barcode molecules include gene-specific or target-specific primer sequences. For example, a barcode molecule of the plurality of barcode molecules can also include a target binding region capable of hybridizing to a specific target nucleic acid associated with the cell, thereby capturing specific targets or analytes of interest. For example, the target binding region capable of hybridizing to a specific target nucleic acid can be a gene-specific primer sequence. The gene-specific primer sequences can be designed based on known sequences of a target nucleic acid of interest. The gene-specific primer sequences can span a nucleic acid region of interest, or adjacent (upstream or downstream) of a nucleic acid region of interest.

The length of the gene-specific primer sequence can vary. For example, a gene-specific primer sequence can be, be about, be at least, be at least about, be at most, or be at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. In some embodiments, the gene-specific primer sequence is at least 10 nucleotides in length.

The number of the barcode molecules introduced into a partition comprising a gene-specific primer sequence can vary. For example, the number of barcode molecules introduced into a partition comprising a gene-specific primer sequence can be, be about, be at least, be at least about, be at most, or be at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, or a number or a range between any two of these values. In some embodiments, the barcode molecules introduced into a partition includes a set of different gene-specific primer sequences each capable of binding to a specific target nucleic acid sequence.

The number of different gene-specific primer sequences of the barcode molecules introduced into a partition can vary. For example, the number of different gene-specific primer sequences of the barcode molecules introduced into a partition can be, be about, be at least, be at least about, be at most, or be at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, 20000, 50000, 1000000, or a number or a range between any two of these values.

The number of target nucleic acids of interest (e.g. genes of interest) that the barcode molecules introduced into a partition are capable of binding can vary. For example, the number of target nucleic acids of interest (e.g. genes of interest) the barcode molecules introduced into a partition are capable of binding can be, be about, be at least, be at least about, be at most, or be at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, 20000, 50000, 1000000, or a number or a range between any two of these values. In some embodiments, one barcode molecule introduced into a partition can bind to a molecule (or a copy) of a target nucleic acid. Barcode molecules introduced into a partition can bind to molecules (or copies) of a target nucleic acid or a plurality of target nucleic acids.

In some embodiments, the barcode molecules of the plurality of barcode molecules each include a poly(dT) sequence, a gene-specific primer sequence, and/or both. The poly(dT) sequence and the gene-specific primer sequence can be on a same barcode molecule or different barcode molecules of the plurality of barcode molecules introduced into a partition.

The ratio of the number of barcode molecules introduced into a partition comprising a poly(dT) sequence and the number of barcode molecules introduced into a partition comprising a gene-specific primer sequence can vary. For example, the ratio can be, be about, be at least, be at least about, be at most, be at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1.65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53.1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99.1, 100:1, or a number or a range between any two of these values.

In some embodiments, a barcode molecule (or each barcode molecule of the plurality of barcode molecules) includes a template switching oligonucleotide (TSO). A primer comprising a target binding region, such as a poly(dT) sequence, can hybridize to a target nucleic acid (e.g., an mRNA) and be extended by, for example, reverse transcription to generate an extended primer comprising a reverse complement of the target nucleic acid, or a portion thereof (e.g., cDNA). The extended primer or cDNA can be further extended to include the reverse complement of a TSO oligonucleotide or barcode molecule. The resulting barcoded nucleic acid includes the barcodes of the barcode molecule on the 3′-end.

In some embodiments, a barcode molecule does not include a TSO. A barcode molecule comprising a target binding region, such as a poly(dT) sequence, can hybridize to a target nucleic acid (e.g., an mRNA) and be extended by, for example, reverse transcription to generate an extended primer comprising a reverse complement of the target nucleic acid, or a portion thereof (e.g., cDNA). The extended primer or cDNA can be further extended to include the reverse complement of a template switching oligonucleotide. The resulting barcoded nucleic acid includes the barcodes of the barcode molecule on the 5′-end. The resulting barcoded nucleic acid (e.g., extended cDNA) can include a PCR primer binding sequence introduced in the reverse complement of the template switching oligonucleotide.

A TSO is an oligonucleotide that hybridizes to untemplated C nucleotides added by a reverse transcriptase during reverse transcription. The TSO can hybridize to the 3′ end of a cDNA molecule. The TSO can include one or more nucleotides with guanine (G) bases on the 3′-end of the TSO, with which the one or more cytosine (C) bases added by a reverse transcriptase to the 3′-end of a cDNA can hybridize. The series of G bases can include 1G base, 2 G bases, 3 G bases, 4 G bases, 5 G bases or more than 5 G bases. The series of G bases can be ribonucleotides. The reverse transcriptase can further extend the cDNA using the TSO as the template to generate a barcoded cDNA comprising the TSO. The length of a TSO can vary. For example, a TSO can be, be about, be at least, be at least about, be at most, or be at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

The number of the barcode molecules introduced into a partition comprising a TSO can vary. In some embodiments, the number of barcode molecules introduced into a partition comprising a TSO is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, or a number or a range between any two of these values.

The TSO of the barcode molecules introduced into a partition can be identical. In some embodiments, the TSO of the barcode molecules introduced into a partition is different. The percentage of the barcode molecules of the plurality of barcode molecules introduced into a partition with an identical TSO sequence can be different in different embodiments. In some embodiments, the percentage of the barcode molecules of the plurality of barcode molecules introduced into a partition with an identical TSO sequence is, is about, is at least, is at least about, is at most, is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 100%, or a number or a range between any two of these values.

### Barcoding Target Nucleic Acids

The method described herein can include barcoding target nucleic acids associated with a cell in the partition (e.g., microwell) using the barcode molecules to generate a barcoded nucleic acids (e.g., target nucleic acids each hybridized with a barcode molecule, single-stranded barcoded nucleic acids, or double-stranded barcoded nucleic acids).

The method can, in some embodiments, further includes releasing the plurality of target nucleic acids associated with the cell in the partition prior to barcoding the plurality of target nucleic acids. In some embodiments, releasing the plurality of target nucleic acids associated with a cell (e.g., a single cell in a microwell of the plurality of microwells) includes lysing the cell using a lysis agent. For example, prior to analyzing (e.g., barcoding) the target nucleic acids, the method can include lysing the cells, thereby releasing the plurality of target nucleic acids from the cell. Lysis agents can be contacted with the cells or cell suspension concurrently, or immediately after the introduction of the cells into the partitions (e.g., microwells) and before the barcoding. Non-limiting examples of lysis agents include bioactive reagents, such as lysis enzymes, or surfactant based lysis solutions including non-ionic surfactants (e.g., Triton X-100 and Tween 20) and ionic surfactants (eg., sodium dodecyl sulfate (SDS)). Lysis methods including, but not limited to, thermal, acoustic, electrical, or mechanical cellular disruption can also be used.

### First Strand Synthesis and Single-Stranded Barcoded Nucleic Acids

In some embodiments, barcoding the plurality of target nucleic acids includes a reverse transcription reaction, for example, to generate a plurality of barcoded nucleic acids comprising complementary deoxyribonucleic acids (cDNAs). In some embodiments, barcoding the plurality of target nucleic acids includes hybridizing the target binding sequence of the barcode molecule to a target nucleic acid of the plurality of target nucleic acids. For example, the target binding sequence can include a poly(dT) sequence and/or a sequence capable of hybridizing to the target nucleic acid, such as a sequence comprising a target specific sequence. In some embodiments, the target binding sequence of the barcode molecule includes a poly(dT) sequence, and barcoding the plurality of target nucleic acids includes hybridizing the poly(dT) sequence of the target binding sequence to a poly(A) sequence of a target nucleic acid of the plurality of target nucleic acids.

In some embodiments, barcoding the plurality of target nucleic acids includes extending the plurality of barcode molecules using the plurality of target nucleic acids as templates to generate the plurality of barcoded nucleic acids comprising a plurality of single-stranded barcoded nucleic acids. In some embodiments, the plurality of single-stranded barcoded nucleic acids can be hybridized to the plurality of target nucleic acids.

In some embodiments, barcoding target nucleic acids associated with a cell in the partition can include extending the barcode molecules using the target nucleic acids as templates to generate partially single-stranded/partially double-stranded barcoded nucleic acids hybridized to the target nucleic acids in the partition (or after target nucleic acids hybridized with barcode molecules are pooled). The partially single-stranded/partially double-stranded barcoded nucleic acids hybridized to target nucleic acids can be separated by denaturation (e.g., heat denaturation or chemical denaturation using for example, sodium hydroxide) to generate single-stranded barcoded nucleic acids of the plurality of barcoded nucleic acids. The single-stranded barcoded nucleic acids can include a barcode molecule and an oligonucleotide complementary to the target nucleic acids. In some embodiments, the single-stranded barcoded nucleic acids are generated by reverse transcription using a reverse transcriptase. In some embodiments, the single-stranded barcoded nucleic acids is generated by using a DNA polymerase.

In some embodiments, the method further includes introducing a plurality of TSO into the partition (e.g., microwell). Barcoding the plurality of target nucleic acids can include extending the plurality of barcode molecules using the plurality of target nucleic acids and the plurality of template switching oligonucleotides as templates to generate the plurality of barcoded nucleic acids comprising a plurality of single-stranded barcoded nucleic acids.

For example, the single-stranded barcoded nucleic acids can be cDNA produced by extending a barcode molecule using a target RNA associated with the cell as a template. The single-stranded barcoded nucleic acids can be further extended using a TSO. The TSO can be introduced into the partitions together with the reverse transcription reagents. For example, a reverse transcriptase can be used to generate a cDNA by extending a barcode molecule hybridized to an RNA. After extending the barcode molecule to the 5′-end of the RNA, the reverse transcriptase can add one or more nucleotides with cytosine (C) bases (e.g. two or three) to the 3′-end of the cDNA. The TSO can include one or more nucleotides with guanine (G) bases (e.g. two or more) on the 3′-end of the TSO. The nucleotides with G bases can be ribonucleotides. The G bases at the 3′-end of the TSO can hybridize to the cytosine bases at the 3′-end of the cDNA. The reverse transcriptase can further extend the cDNA using the TSO as the template to generate a cDNA with the reverse complement of the TSO sequence on its 3′-end. The barcoded nucleic acid can include the barcode sequences (e.g., particle barcode sequence and molecular barcode sequence (e.g., UMI)) on the 5′-end and a TSO sequence at its 3′-end.

In some embodiments, barcoding the target nucleic acids includes extending the barcode molecules using the target nucleic acids as templates and the barcode molecules as TSO to generate single-stranded barcoded nucleic acids that are hybridized to the target nucleic acids. In some embodiments, the present method further includes introducing a plurality of extension primers to the partition. Barcoding the plurality of target nucleic acids can include extending the plurality of extension primers using the plurality of target nucleic acids as templates and the plurality of barcode molecules as template switching oligonucleotides to generate the plurality of barcoded nucleic acids comprising a plurality of single-stranded barcoded nucleic acids.

In some embodiments, the barcode molecules can include TSO. For example, the plurality of target nucleic acids can includes poly-adenylated messenger ribonucleic acid (mRNA), and the extension primers can include a poly(dT) sequence. The extension primers can be extended using the target nucleic acids as a template. For example, a reverse transcriptase can be used to generate a cDNA by extending an extension primer hybridized to an RNA. After extending the extension primers to the 5′-end of the RNA, the reverse transcriptase can add one or more C bases (e.g. two or three) to the 3′-end of the cDNA. The TSO or barcode molecule can include one or more G bases (e.g. two or more) on the 3′-end of the TSO. The nucleotides with guanine bases can be ribonucleotides. The G bases at the 3′-end of the TSO or barcode molecule can hybridize to the cytosine bases at the 3′-end of the cDNA. The reverse transcriptase can switch template from the mRNA to the TSO or barcode molecule. The reverse transcriptase can further extend the cDNA using the TSO or barcode molecule as the template to generate a cDNA further comprising the reverse complement of the TSO or barcode molecule. In this case, the barcode sequences (e.g., particle barcode sequence and molecular barcode sequence (e.g., UMI)) are on the 3′-end of the generated cDNA.

In some embodiments, each of the plurality of single-stranded barcoded nucleic acids is hybridized to one of the plurality of target nucleic acids and one of the plurality of template switching oligonucleotides in the partition (e.g, microwell).

The single-stranded barcoded nucleic acids can be separated from the template target nucleic acids by digesting the template target nucleic acids (e.g, using RNase), by chemical treatment (e.g., using sodium hydroxide), by hydrolyzing the template target nucleic acids, or via a denaturation or melting process by increasing the temperature, adding organic solvents, or increasing pH. Following the melting process, the target nucleic acids can be removed (e.g. washed away) and the single-stranded barcoded nucleic acids can be retained in the partition (e.g. through attachment to the partitions or through attachments to particles which can be retained in the partitions). In some embodiments, the method further includes removing the plurality of target nucleic acids and the plurality of template switching oligonucleotides hybridized to the single-stranded barcoded nucleic acids. In some embodiments, removing the plurality of target nucleic acids includes denaturation, thermal denaturation, digesting, or hydrolyzing the plurality of target nucleic acids.

In some embodiments, each of the plurality of single-stranded barcoded nucleic acid includes a sequence of a barcode molecule of the plurality of barcode molecules (e.g., an actual sequence of the barcode molecule), a sequence of a target nucleic acid of the plurality of target nucleic acids (e.g., a reverse complement of the target nucleic acid), a sequence of a template switching oligonucleotide of the plurality of template switching oligonucleotides (e.g., a reverse complement of the template switching oligonucleotide), and/or a sequence of an extension primer of the plurality of extension primers (e.g., an actual sequence of the extension primer).

### Second Strand Synthesis, Amplification, and Double-Stranded Barcoded Nucleic Acids

The method can further include amplifying the plurality of barcoded nucleic acids to generate a plurality of double-stranded barcoded nucleic acids in the partition (e.g., microwell) using the single-stranded barcoded nucleic acids as templates. The amplifying step can be used to amplify the product of first strand synthesis. In some embodiments, each of the plurality of barcode molecules can include a primer sequence. The primer sequence can include, for example, a PCR primer sequence. Amplifying the plurality of barcoded nucleic acids can include amplifying the plurality of barcoded nucleic acids using the primer sequences in single-stranded barcoded nucleic acids of the plurality of single-stranded barcoded nucleic acids, or products thereof.

For example, barcoding target nucleic acids associated with the cell in the partition (e.g., microwell) can include amplifying the barcoded nucleic acids (such as a single-stranded barcoded nucleic acid, or a cDNA generated by using a barcode molecule as disclosed herein). The amplification can include generating barcoded nucleic acids comprising double-stranded barcoded nucleic acids in the partition using the single-stranded barcoded nucleic acids as templates. The double-stranded barcoded nucleic acids can be generated from the single-stranded barcoded nucleic acids retained in the partition using, for example, second-strand synthesis or one-cycle PCR Amplification of the barcoded nucleic acids can include additional cycles of PCR reactions.

The generated double-stranded barcoded nucleic acid can be denaturized or melted to generate two single-stranded barcoded nucleic acids: one single-stranded barcoded nucleic acid retained in the partition (e.g., attached to the particle) and the other single-stranded barcoded nucleic acid released into the solution from the retained single-stranded barcoded nucleic acid that can then be pooled to provide a pooled mixture outside the partitions. Both single-stranded barcoded nucleic acids (e.g. retained in the partitions or pooled outside the partitions) can have a sequence comprising a sequence of a barcode molecule (e.g. particle barcode sequence and molecular barcode sequence (e.g., UMI)) and a sequence of a target nucleic acid or a reverse complement thereof.

### Pooling

The methods disclosed herein can include pooling the plurality of barcoded nucleic acids, or products thereof, in each of the plurality of partitions to generate pooled barcoded nucleic acids. Subjecting the plurality of barcoded nucleic acids, or products thereof, to sequencing can include subjecting the pooled barcoded nucleic acids, or products thereof, to sequencing. In some embodiments, pooling the plurality of barcoded nucleic acids, or products thereof, includes pooling the plurality of double-stranded barcoded nucleic acids in each of the plurality of partitions to generate the pooled barcoded nucleic acids. For example, the method can include pooling the barcoded nucleic acids after barcoding the target nucleic acids and before sequencing the barcoded nucleic acids to obtain pooled barcoded nucleic acids.

In some embodiments, pooling barcoded nucleic acids occurs after generating double-stranded barcoded nucleic acids (e.g., after second strand synthesis) or after generating amplified barcoded nucleic acids. The amplified barcoded nucleic acids can be subject to sequencing library construction prior to sequencing. In some embodiments, synthesis of single-stranded barcoded nucleic acids and double-stranded barcoded nucleic acids occur after the pooling of target nucleic acids hybridized with barcode molecules.

In some embodiments the barcode molecules are attached to particles, only single-stranded barcoded nucleic acids released into bulk (e.g., after amplification of the barcoded nucleic acids) are collected by pooling, and the particles are not pooled (e.g. not removed from the partitions) but retained in the partitions (e.g. by an external magnetic field applied on magnetic beads), thereby allowing one to trace the origin of the pooled barcoded nucleic acids, for example, to its original location in the partitions.

The pooled barcoded nucleic acids can be single-stranded or double-stranded (e.g. generated from the single-stranded pooled barcoded nucleic acids by PCR amplification). The pooled barcoded nucleic acids (e.g. amplified barcoded cDNA) can be purified, and optionally further amplified, prior to sequencing library construction. The pooled barcoded nucleic acids with desired length can be selected.

### Sequencing Library Construction

The barcoded nucleic acids (e.g. pooled barcoded nucleic acids) can be further processed prior to sequencing to generate processed barcoded nucleic acids. For example, the method herein can include amplification of barcoded nucleic acids, fragmentation of amplified barcoded nucleic acids, end repair of fragmented barcoded nucleic acids, A-tailing of fragmented barcoded nucleic acids that have been end-repaired (e.g., to facilitate ligation to adapters), and attaching (e.g. by ligation and/or PCR) with a second sequencing primer sequence (e.g. a Read 2 sequence), sample indexes (e.g. short sequences specific to a given sample library), and/or flow cell binding sequences (e.g. P5 and/or P7). Additional PCR amplification can also be performed. This process can also be referred to as sequencing library construction.

PCR amplification can be carried out to generate sufficient mass for the subsequent library construction processes. In some embodiments, the present method includes performing a polymerase chain reaction in bulk on the pooled barcoded nucleic acids, or the fragmented barcoded nucleic acids, to generate amplified barcoded nucleic acids. For example, the method can include performing a polymerase chain reaction in bulk, subsequent to the pooling, on the pooled barcoded nucleic acids, thereby generating amplified barcoded nucleic acids. In some embodiments, performing the polymerase chain reaction in bulk is subsequent to fragmenting the pooled barcoded nucleic acids. The amplification for library preparation can be a separate process from the amplification of the first strand barcoded nucleic acid generated by, for example, the RT reaction as described herein.

In some embodiments, the method includes fragmenting the pooled barcoded nucleic acids to generate fragmented barcoded nucleic acids to generate fragmented barcoded nucleic acids prior to subjecting the plurality of barcoded nucleic acids, or products thereof, to sequencing. For example, the method can include fragmenting (e.g., via enzymatic fragmentation, mechanical force, chemical treatment, etc.) the pooled barcoded nucleic acids to generate fragmented barcoded nucleic acids. Fragmentation can be carried out by any suitable process such as physical fragmentation, enzymatic fragmentation, or a combination of both. For example, the barcoded nucleic acids can be sheared physically using acoustics, nebulization, centrifugal force, needles, or hydrodynamics. The barcoded nucleic acids can also be fragmented using enzymes, such as restriction enzymes and endonucleases.

Fragmentation yields fragments of a desired size for subsequent sequencing. The desired sizes of the fragmented nucleic acids are determined by the limitations of the next generation sequencing instrumentation and by the specific sequencing application as will be understood by a person skilled in the art. For example, when using Illumina technology, the fragmented nucleic acids can have a length of between about 50 bases to about 1,500 bases. In some embodiments, the fragmented barcoded nucleic acids have about 100 bp to 700 bp in length.

Fragmented barcoded nucleic acids can undergo end-repair and A-tailing (to add one or more adenine bases) to form an A overhang. This A overhang allows adapter containing one or more thymine overhanging bases to base pair with the fragmented barcoded nucleic acids.

Fragmented barcoded nucleic acids can be further processed by adding additional sequences (e.g. adapters) for use in sequencing based on specific sequencing platforms. Adapters can be attached to the fragmented barcoded nucleic acids by ligation using a ligase and/or PCR. For example, fragmented barcoded nucleic acids can be processed by adding a second sequencing primer sequence. The second sequencing primer sequence can include a Read 2 sequence. An adapter comprising the second primer sequence can be ligated to the fragmented barcoded nucleic acids after, for example, end-repair and A tailing, using a ligase. The adaptor can include one or more thymine (T) bases that can hybridize to the one or more A bases added by A tailing. An adaptor can be, for example, partially double-stranded or double stranded. In some embodiments, the amplified barcoded nucleic acids include a sequencing primer sequence.

The adapter can also include platform-specific sequences for fragment recognition by specific sequencing instrument. In some embodiments, the amplified barcoded nucleic acids include a sequence for attaching the amplified barcoded nucleic acids to a flow well. For example, the amplified barcoded nucleic acids can include an adapter that includes a sequence for attaching the fragmented barcoded nucleic acids to a flow well of Illumina platforms, such as a P5 sequence, a P7 sequence, or a portion thereof. Different adapter sequences can be used for different next generation sequencing instrument as will be understood by a person skilled in the art.

The adapter can also contain sample indexes to identify samples and to permit multiplexing. Sample indexes enable multiple samples to be sequenced together (i.e. multiplexed) on the same instrument flow cell as will be understood by a person skilled in the art. Adapters can include a single sample index or a dual sample indexes depending on the implementations such as the number of libraries combined and the level of accuracy desired.

In some embodiments, the amplified barcoded nucleic acids generated from sequencing library construction can include a P5 sequence, a sample index, a Read 1 sequence, a particle barcode sequence, a molecular barcode sequence (e.g., UMI), a poly(dT) sequence, a target biding region, a sequence of a target nucleic acid or a portion thereof, a Read 2 sequence, a sample index, and/or a P7 sequence (e.g., from 5′-end to 3′-end). In some embodiments, the amplified barcoded nucleic acids can include a P5 sequence, a sample index, a Read 1 sequence, a particle barcode sequence, a molecular barcode sequence (e.g., UMI), a sequence of a template switching oligonucleotide, a sequence of a target nucleic acid or a portion thereof, a Read 2 sequence, a sample index, and/or a P7 sequence (e.g, from 5′-end to 3′-end).

Sequencing the barcoded nucleic acids, or products thereof, can include sequencing products of the barcoded nucleic acids. Products of the barcoded nucleic acids can include the processed nucleic acids generated by any step of the sequencing library construction process, such as amplified barcoded nucleic acids, fragmented barcoded nucleic acids, fragmented barcoded nucleic acids comprising additional sequences such as the second sequencing primer sequence and/or adapter sequences described herein.

### Sequencing Barcoded Nucleic Acids

The method disclosed herein can include sequencing the barcoded nucleic acids or products thereof to obtain nucleic acid sequences of the barcoded nucleic acids. The barcoded nucleic acids generated by the method disclosed herein can include barcoded nucleic acids pooled, from each partition, into a pooled mixture outside the partitions. The barcoded nucleic acids retained in a partition and the pooled barcoded nucleic acids in a pooled mixture outside the partitions can be sequenced using a same or different sequencing techniques.

In some embodiments, sequencing the plurality of barcoded nucleic acids or products thereof includes sequencing the pooled barcoded nucleic acids to obtain nucleic acid sequences of the pooled barcoded nucleic acids. As used herein, a “sequence” can refer to the sequence, a complementary sequence thereof (e.g., a reverse, a compliment, or a reverse complement), the full-length sequence, a subsequence, or a combination thereof. The nucleic acids sequences of the pooled barcoded nucleic acids can each include a sequence of a barcode molecule (e.g., the particle barcode sequence and the molecular barcode sequence (e.g., UMI)) and a sequence of a target nucleic acid associated with the cell or a reverse complement thereof.

Pooled barcoded nucleic acids can be sequenced using any suitable sequencing method identifiable. For example, sequencing the pooled barcoded nucleic acids can be performed using high-throughput sequencing, pyrosequencing, sequencing-by-synthesis, single-molecule sequencing, nanopore sequencing, sequencing-by-ligation, sequencing-by-hybridization, next generation sequencing, massively-parallel sequencing, primer walking, and any other sequencing methods known in the art and suitable for sequencing the barcoded nucleic acids generated using the methods herein described.

### Analysis

Method disclosed herein can include determining a profile of the cells, for example from the sequence of the barcode nucleic acids. For example, the obtained nucleic acid sequences of the plurality of barcoded nucleic acids (e.g. nucleic acid sequences of pooled barcoded nucleic acids) can be subjected to any downstream post-sequencing data analysis as will be understood by a person of skill in the art. The sequence data can undergo a quality control process to remove adapter sequences, low-quality reads, uncalled bases, and/or to filter out contaminants. The high-quality data obtained from the quality control can be mapped or aligned to a reference genome or assembled de novo.

Profile analysis, for example gene expression quantification and differential expression analysis, can be carried out to identify genes whose expression differs in different cells. Barcoded nucleic acids from a cell can have an identical particle barcode sequence in the sequencing data and can be identified. Barcoded nucleic acids from different cells can have different particle barcode sequences in the sequencing data and can be identified. Barcoded nucleic acids with an identical particle barcode sequence, an identical target sequence, and different molecular barcode sequences in the sequencing data can be quantified and used to determine the expression of the target.

A profile determined from the barcoded nucleic acids can include, for example, an expression profile, a transcription profile, an omics profile, a multi-omics profile, or a combination thereof. In some embodiments, the profile includes a single omics profile, such as a transcriptome profile. In some embodiments, the profile includes a multi-omics profile, which can include profiles of genome (e.g. a genomics profile), proteome (e.g. a proteomics profile), transcriptome (e.g. a transcriptomics profile), epigenome (e.g. an epigenomics profile), metabolome (e.g. a metabolomics profile), and/or microbiome (e.g. a microbiome profile). In some embodiments, the multi-omics profile includes a genomics profile, a proteomics profile, a transcriptomics profile, an epigenomics profile, a metabolomics profile, a chromatics profile, a protein expression profile, a cytokine secretion profile, or a combination thereof.

In some embodiments, the profile includes an expression of a target nucleic acid of the plurality of target nucleic acids. For example, the expression of the target nucleic acid can include an abundance of the target nucleic acid. The abundance of the target nucleic acid can include an abundance of molecules of the target nucleic acid barcoded using the barcode molecules. The abundance of the molecules of the target nucleic acid can include a number of occurrences of the molecules of the target nucleic acid. In some embodiments, the number of occurrences of the molecules of the target nucleic acid is, is indicated by, or is determined using, a number of the barcoded nucleic acids comprising a sequence of the target nucleic acid and different molecular barcode sequences in the sequences of the barcoded nucleic acids.

For example, the profile can include an RNA expression profile and/or a protein expression profile. The expression profile can include an RNA expression profile, an mRNA expression profile, and/or a protein expression profile. A profile can also be a profile of one or more target nucleic acids (e.g. gene markers) or a selection of genes associated with the cell.

### EXAMPLES

Some aspects of the embodiments discussed above are disclosed in further detail in the following example, which are not in any way intended to limit the scope of the present disclosure.

### Example 1

### Microfluidic Chip and Single Cell Analysis

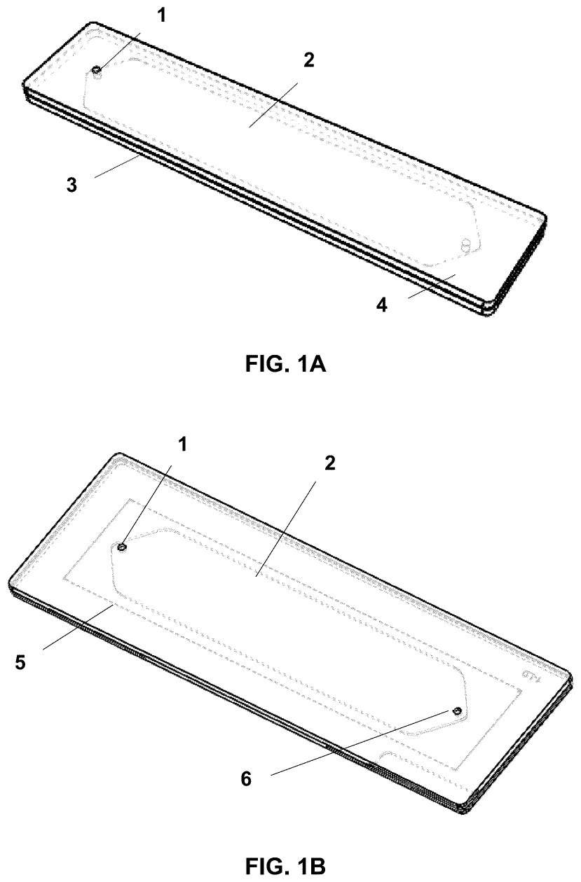

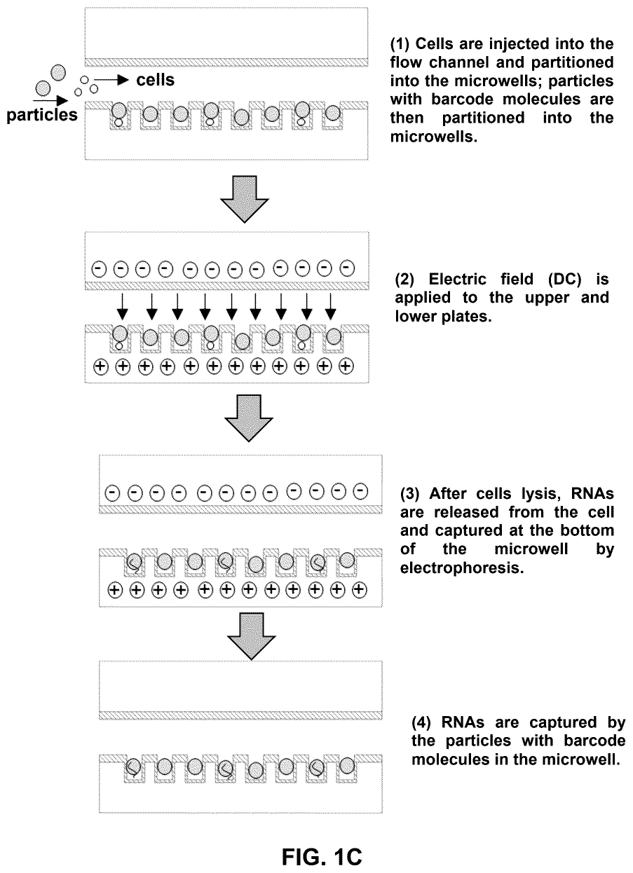

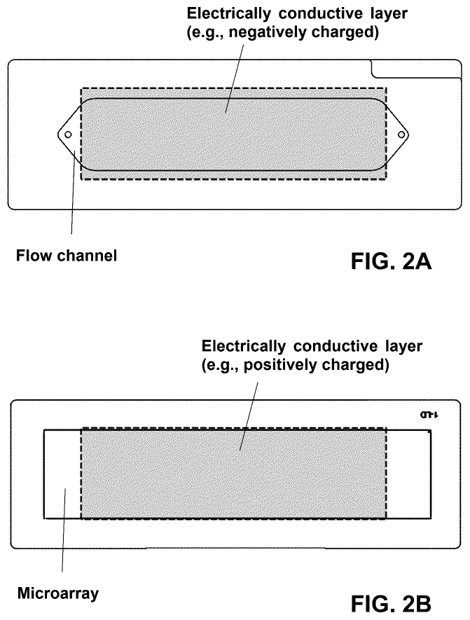

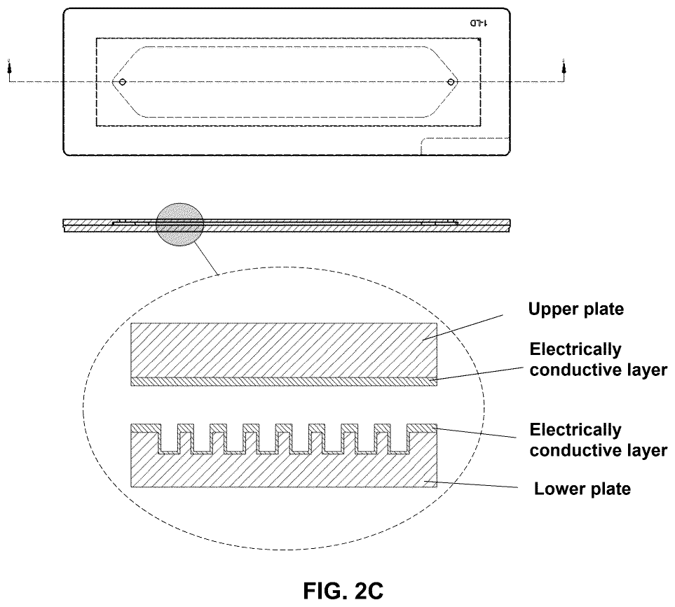

An exemplary microfluidic chip is provided, which includes an upper plate, a lower plate, a flow channel (with tapered ends), a microwell array, a sample inlet, and a sample outlet (FIGS. 1A and 1B). The chip can have electronically conductive layers disposed on the upper and lower plates (FIG. 2C). The electronically conductive layer on the upper plate can cover the flow channel (FIG. 2A). The electronically conductive layer on the lower plate can cover the microwell array (FIG. 2B).

As illustrated in FIG. 1C, cells and beads with attached barcode molecules are injected into the flow channel of the chip, and are partitioned into the microwells. An electric field is applied in the direction perpendicular to plates. A positive potential is applied to the microwell surface on the lower plate. A negative potential is applied to the lower surface of the upper plate. A lysis agent is then introduced, and RNA molecules are released from the cell following cell lysis. Since the RNA molecules are negatively charged, they are restricted or immobilized in the microwell in the perpendicular electric field. The RNA molecules can then be captured and barcoded by the barcode molecules attached to the bead. Once the barcoding reaction is complete, the electric field is removed, and the beads can be collected. Thus, the present microfluidic chip design combining electrophoresis function and microwell array can improve the efficiency in RNA capture and analysis.

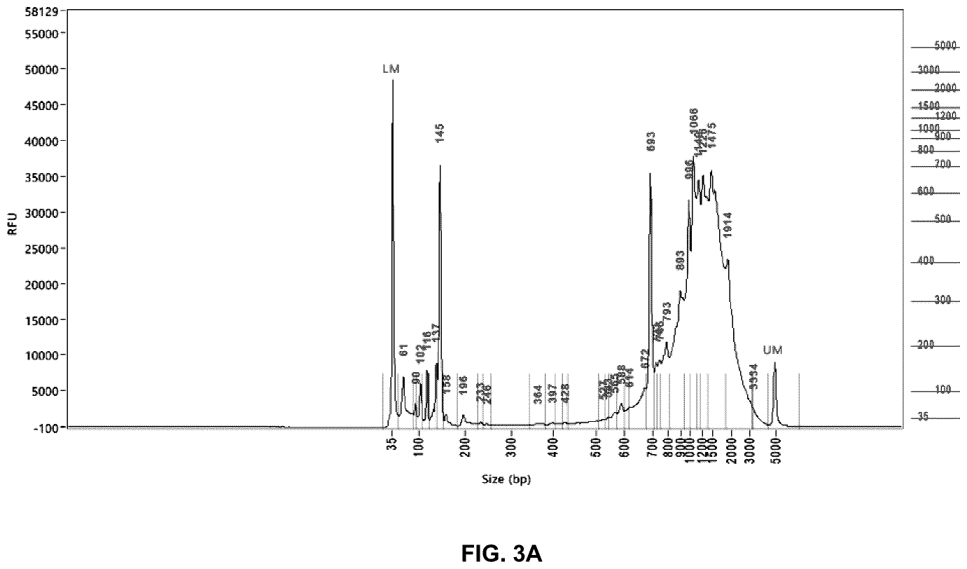

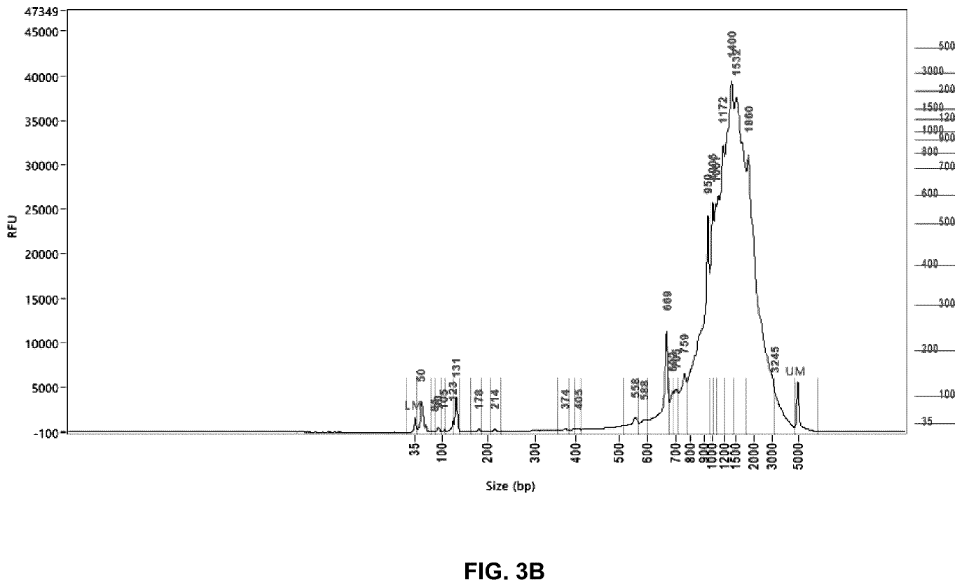

Cells from mouse testicle are analyzed using a control device (FIG. 3A) and the present microfluidic chip with applied electric potential (1.6V, 15 min, FIG. 3B). The results are summarized in Table 1.

TABLE 1
Results for cells from mouse testicle

| | Control | Exp |
|---|---|---|
| | (cDNA Qubit 12.4) | (cDNA Qubit 54.2) |
| Estimated Number of Cells | 4,723 | 7,703 |
| Fraction Reads in Cells | 56.98% | 62.46% |
| Mean Reads per Cell | 28,834 | 25,777 |
| Median UMI per Cell | 4,956 | 6,207 |
| Total Genes | 29,888 | 31,202 |
| Median Genes per Cell | 2,150 | 2,435 |
| Saturation | 33.59% | 33.54% |

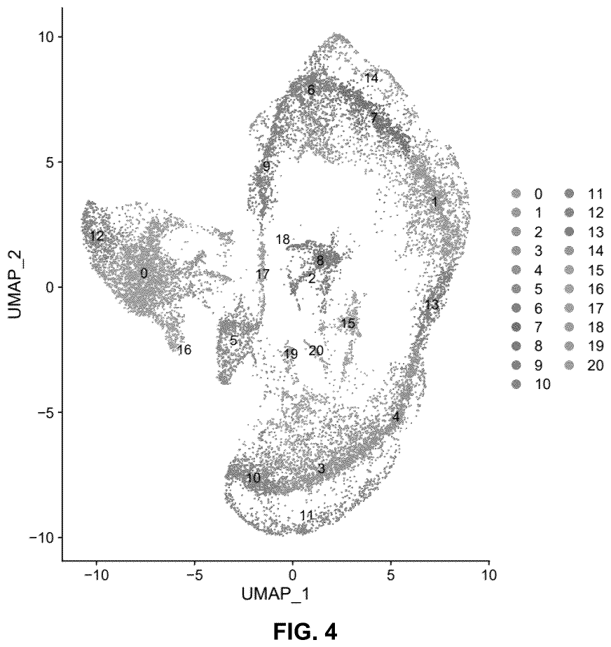

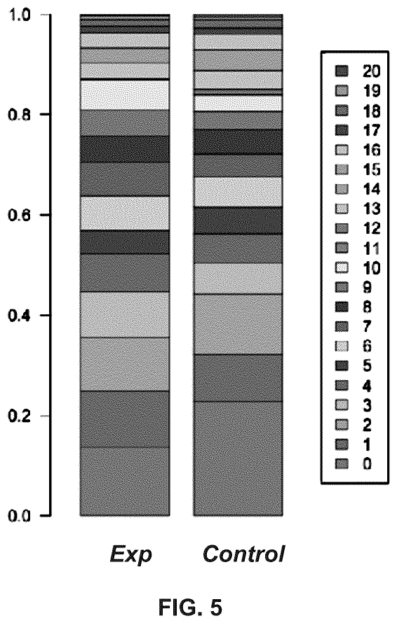

Clusters of cells from mouse testicle are identified using the present microfluidic chip (FIG. 4). The results obtained in the presence (Exp) or absence (Control) of applied electric potential are compared for the analysis of cells from mouse testicle (FIG. 5).

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A microfluidic device comprising:
a first layer with a lower surface comprising a first electrically conductive layer disposed thereon;
a second layer with a microwell array disposed thereon and comprising at least 100 microwells, wherein a upper surface of the array of microwells comprises a second electrically conductive layer disposed thereon and not in contact with the first electrically conductive layer;
a first electric terminal and a second electric terminal in electrical communication with the first electrically conductive layer and the second electrically conductive layer, respectively;
a flow channel formed by the first layer and the second layer; and
an inlet and an outlet in fluid communication with the flow channel.

2. The microfluidic device of claim 1, wherein the first layer is in direct contact with the second layer.

3. The microfluidic device of claim 1, wherein the microfluidic device comprises a third layer between the first layer and the second layer, and the third layer is in direct contact with the first layer and the second layer.

4. The microfluidic device of any one of claims 1-3, wherein the first layer is a cover plate, and wherein the second layer is a bottom plate.

5. The microfluidic device of any one of claims 1-4, wherein the microfluidic device comprises a cover plate and/or a bottom plate.

6. The microfluidic device of any one of claims 1-5, wherein the first electrically conductive layer is rectangular in shape, wherein
the first electrically conductive layer is 5 cm² in size, and/or
50% of the lower surface of the first layer comprises the first electrically conductive layer.

7. The microfluidic device of any one of claims 1-6, wherein the second electrically conductive layer is rectangular in shape, wherein
the second electrically conductive layer is 5 cm² in size, and/or
50% of the upper surface of the second layer comprises the second electrically conductive layer.

8. The microfluidic device of any one of claims 1-7, wherein a shape of the first electrically conductive layer and a shape of the second electrically conductive layer are identical.

9. The microfluidic device of any one of claims 1-8, wherein a size of the lower surface of the first layer and a size of the upper surface of the second layer are identical.

10. The microfluidic device of any one of claims 1-9, wherein the first electric terminal is on an outer surface of the microfluidic device, wherein
the first electric terminal extrudes from an outer surface of the microfluidic device, and/or
the first electric terminal is recessed into an outer surface of the microfluidic device.

11. The microfluidic device of any one of claims 1-10, wherein the second electric terminal is on an outer surface of the microfluidic device, wherein
the second electric terminal extrudes from an outer surface of the microfluidic device, and/or
the second electric terminal is recessed into an outer surface of the microfluidic device.

12. The microfluidic device of any one of claims 1-11, further comprising a first indicator that indicates that the first electric terminal is a negative electric terminal, wherein the first indicator is on an outer surface of the microfluidic device.

13. The microfluidic device of any one of claims 1-12, further comprising a second indicator that indicates that the second electric terminal is a positive electric terminal, wherein the second indicator is on an outer surface of the microfluidic device.

14. The microfluidic device of any one of claims 1-13, wherein the flow channel comprises a rectangular section.

15. The microfluidic device of any one of claims 1-14, wherein
the flow channel comprises a first tapered end which is triangular in shape, and/or
the inlet is at the first tapered end.

16. The microfluidic device of any one of claims 1-15, wherein
the flow channel comprises a second tapered end which is triangular in shape, and/or
the outlet is at the second tapered end.

17. The microfluidic device of any one of claims 1-16, wherein
the inlet comprises a hole in the first layer or the second layer, and/or
the outlet comprises a hole in the first layer or the second layer.

18. The microfluidic device of any one of claims 1-17, wherein the flow channel comprises an outer surface of the first electrically conductive layer.

19. The microfluidic device of any one of claims 1-18, wherein the flow channel comprises an outer surface of the second electrically conductive layer.

20. The microfluidic device of any one of claims 11-19, wherein a distance between the first layer and the second layer is 1 μm to 100 μm.

21. The microfluidic device of any one of claims 1-20, wherein a thickness of the first electrically conductive layer is 0.1 μm to 5 μm.

22. The microfluidic device of any one of claims 1-21, wherein a thickness of the second electrically conductive layer is 0.1 μm to 5 μm.

23. The microfluidic device of any one of claims 1-22, wherein a width of the microwell is 10 μm to 200 μm.

24. The microfluidic device of any one of claims 1-23, wherein a length of the microwell is 10 μm to 200 μm.

25. The microfluidic device of any one of claims 1-24, wherein a depth of the microwell is 5 μm to 500 μm.

26. The microfluidic device of any one of claims 1-25, wherein the microwell has a circular, elliptical, square, rectangular, triangular, or hexagonal shape.

27. A system comprising:
a holder of a microfluidic device of any one of claims 1-26;
an inlet fluidic interface for fluidic communication with the inlet of the microfluidic device;
an outlet fluidic interface for fluidic communication with the outlet of the microfluidic device;
one or more pumps for introducing one or more fluids into the microfluidic device via the inlet fluidic interface and the inlet of the microfluidic device;
a first electric interface for connecting with the first electric terminal of the microfluidic device; and
a second electric interface for connecting with the second electric terminal of the microfluidic device.

28. A kit comprising
a microfluidic device of any one of claims 1-26; and
instructions for using the microfluidic device.

29. The kit of claim 28, wherein the instructions comprise instructions for applying a voltage between (i) the first electrical terminal as a negative terminal and (ii) the second electrical terminal as a positive terminal.

30. The kit of any one of claims 28-29, wherein the instructions comprise instructions for using the microfluidic device for single cell sequencing.

31. The kit of claim 30, wherein the single cell sequencing comprises single cell ribonucleic acid (RNA) sequencing or multiomics sequencing.

32. The kit of any one of claims 30-31, wherein the kit comprises one or more reagents for single cell sequencing.

33. A method of analyzing nucleic acids, comprising:
co-partitioning a plurality of cells and a plurality of particles into a plurality of microwells of a microwell array, thereby at least 25% of the plurality of microwells each comprises a single cell of the plurality of cells and a single particle of the plurality of particles;
applying an electric field to the microwell array;
while applying the electric field to the microwell array:
releasing a plurality of target nucleic acids associated with the single cell in a microwell of the plurality of microwells; and
barcoding the plurality of target nucleic acids released to generate a plurality of barcoded nucleic acids; and
analyzing the plurality of barcoded nucleic acids.

34. A method of analyzing nucleic acids, comprising:
co-partitioning a plurality of cells and a plurality of particles into a plurality of microwells of a microwell array, thereby at least 25% of the plurality of microwells each comprises a single cell of the plurality of cells and a single particle of the plurality of particles;
releasing a plurality of target nucleic acids associated with the single cell in a microwell of the plurality of microwells;
applying an electric field to the microwell array;
while applying the electric field to the microwell, barcoding the plurality of target nucleic acids released to generate a plurality of barcoded nucleic acids; and
analyzing the plurality of barcoded nucleic acids.

35. The method of any one of claims 33-34, wherein the particles each comprises a plurality of barcode molecules, and wherein barcoding the plurality of target nucleic acids released to generate a plurality of barcoded nucleic acids comprises barcoding the plurality of target nucleic acids released using the plurality of barcode molecules of the particle in the microwell to generate a plurality of barcoded nucleic acids

36. A method of analyzing nucleic acids, comprising:
partitioning a plurality of cells into a plurality of microwells of a microwell array, thereby at least 25% of the plurality of microwells each comprises a single cell of the plurality of cells;
releasing a plurality of target nucleic acids associated with the single cell in a microwell of the plurality of microwells;
applying an electric field to the microwell array; and
while applying the electric field to the microwell:
introducing a reaction reagent into the microwell; and
performing a reaction on the target nucleic acids using the reaction reagent.

37. The method of claim 36, wherein the reaction reagent comprises one or more barcoding reagents, and wherein the reaction comprises a barcoding reaction.

38. The method of any one of claims 36-37, wherein the barcoding reagents comprise barcode molecules.

39. The method of any one of claims 36-37, further comprising, after partitioning the plurality of cells into the plurality of microwells and prior to applying the electric field, partitioning a plurality of particles each comprising a plurality of barcode molecules into the plurality of microwells, thereby at least 25% of the plurality of microwells each comprises a single cell of the plurality of cells and a single particle of the plurality of particles.

40. The method of any one of claims 36-39, further comprising analyzing a reaction product of the target nucleic acids generated using the reaction reagent.

41. A method of manipulating nucleic acids, comprising:
partitioning a plurality of cells into a plurality of microwells, thereby at least 25% of the plurality of microwells each comprises a single cell of the plurality of cells;
releasing a plurality of target nucleic acids associated with the single cell in a microwell of the plurality of microwells; and
applying an electric field to the microwell array, thereby controlling a movement of the target nucleic acids.

42. The method of claim 41, further comprising, before releasing the plurality of target nucleic acids, partitioning a plurality of particles into the plurality of microwells, thereby at least 25% of the plurality of microwells each comprises a single cell of the plurality of cells and a single particle of the plurality of particles.

43. A method of analyzing nucleic acids, comprising:
manipulating a plurality of target nucleic acids according to any one of claims 41-42;
barcoding the plurality of target nucleic acids released using a plurality of barcode molecules to generate a plurality of barcoded nucleic acids; and
analyzing the plurality of barcoded nucleic acids.

44. A method of detecting a biomolecule, comprising:
partitioning a plurality of cells into a plurality of microwells of a microwell array, thereby at least 25% of the plurality of microwells each comprises a single cell of the plurality of cells;
introducing a reaction reagent into the plurality of microwells;
applying an electric field to the plurality of microwells, thereby the reaction reagent enters the cell;
within the cell, the reaction reagent interacts with a biomolecule, and
detecting the biomolecule.

45. The method of claim 44, wherein the electric field increases a permeability of a membrane of the cell, thereby the reaction reagent enters the cell after the permeability of the membrane of the cell is increased.

46. The method of any one of claims 44-45, wherein the electric field causes electroporation.

47. The method of any one of claims 44-46, wherein the reaction reagent is a probe capable of capturing the biomolecule.

48. The method of claim 47, wherein the probe is a small molecule compound, a polypeptide, an oligonucleotide, a ribonucleic acid (RNA), a deoxyribonucleic acid (DNA), an oligosaccharide, a sugar, or a combination thereof.

49. A method of analyzing a biomolecule, comprising:
within a partition comprising a biomolecule, applying an electric field, thereby manipulating a movement of the biomolecule within the partition; and
analyzing the biomolecule.

50. The method of claim 44-49, wherein the biomolecule is a polypeptide, a protein, an oligonucleotide, a ribonucleic acid (RNA), a deoxyribonucleic acid (DNA), an oligosaccharide, a sugar, or a combination thereof.

51. The method of claim 49-50, wherein the biomolecule is associated or previously associated with a single cell, optionally wherein the method comprises introducing the single cell into the partition, optionally wherein the method comprises releasing the biomolecule from the single cell.

52. The method of any one of claims 49-51, further comprising introducing a plurality of barcode molecules into the partition.

53. The method of claim 52, further comprising barcoding the biomolecule using the plurality of barcode molecules.

54. The method of any one of claims 49-53, wherein the partition is a droplet or a microwell of a microwell array comprising a plurality of microwells.

55. The method of claim 54, wherein the partition is a microwell of a microwell array comprising a plurality of microwells.

56. The method of any one of claims 33-55, wherein applying the electric field to the microwell is performed before releasing the plurality of target nucleic acids associated with the single cell in the microwell of the plurality of microwells.

57. The method of any one of claims 33-56, wherein releasing the plurality of target nucleic acids comprises: while applying the electric field to the microwell array, releasing the plurality of target nucleic acids associated with the single cell in the microwell of the plurality of microwells.

58. The method of any one of claims 33-57, wherein applying the electric field to the microwell is performed after releasing the plurality of target nucleic acids associated with the single cell in the microwell of the plurality of microwells.

59. The method of any one of claims 33-58, further comprising: while applying the electric field:
introducing a first reaction reagent into the microwell; and
performing a first reaction on a content of the single cell in the microwell using the first reaction reagent.

60. The method of claim 59, further comprising: while applying the electric field:
introducing a second reaction reagent into the microwell; and
performing a second reaction on a content of the single cell in the microwell using the second reaction reagent.

61. The method of any one of claims 33-60, wherein each of the plurality of particles comprises a plurality of barcode molecules, and wherein each barcode molecule of the plurality of barcode molecules comprises a molecular barcode sequence, a particle barcode sequence, and optionally a target binding sequence.

62. The method of any one of claims 33-61, wherein, as a result of the partitioning, at least 90% of the plurality of microwells each comprises at most one of the plurality of cells.

63. The method of any one of claims 33-62, wherein the electric field restricts the movement the plurality of target nucleic acids or biomolecule in the partition.

64. The method of any one of claims 33-63, wherein the microwell comprises an open end and a closed end facing the open end, wherein the electric field is applied in a direction from the opened end to the closed end, or an opposite direction thereof.

65. The method of any one of claims 33-64, wherein barcoding the plurality of target nucleic acids comprises extending the plurality of barcode molecules using the plurality of target nucleic acids as templates to generate the plurality of barcoded nucleic acids comprising a plurality of single-stranded barcoded nucleic acids, optionally hybridized to the plurality of target nucleic acids.

66. The method of claim 65, further comprising introducing a plurality of template switching oligonucleotides into the microwell, wherein barcoding the plurality of target nucleic acids comprises extending the plurality of barcode molecules using the plurality of target nucleic acids and the plurality of template switching oligonucleotides as templates to generate the plurality of barcoded nucleic acids comprising a plurality of single-stranded barcoded nucleic acids.

67. The method of any one of claims 33-64, further comprising introducing a plurality of extension primers to the microwell, and wherein barcoding the plurality of target nucleic acids comprises extending the plurality of extension primers using the plurality of target nucleic acids as templates and the plurality of barcode molecules as template switching oligonucleotides to generate the plurality of barcoded nucleic acids comprising a plurality of single-stranded barcoded nucleic acids.

68. The method of any one of claims 65-67, wherein each of the plurality of single-stranded barcoded nucleic acids is hybridized to one of the plurality of target nucleic acids and one of the plurality of template switching oligonucleotides in the microwell.

69. The method of any one of claims 65-68, further comprising removing the plurality of target nucleic acids and the plurality of template switching oligonucleotides hybridized to the single-stranded barcoded nucleic acids, wherein removing the plurality of target nucleic acids comprises denaturation, thermal denaturation, digesting, or hydrolyzing the plurality of target nucleic acids.

70. The method of any one of claims 65-69, wherein each of the plurality of single-stranded barcoded nucleic acid comprises a sequence of a barcode molecule of the plurality of barcode molecules, a sequence of a target nucleic acid of the plurality of target nucleic acids, a sequence of a template switching oligonucleotide of the plurality of template switching oligonucleotides, and/or a sequence of an extension primer of the plurality of extension primers.

71. The method of claim 69 or 70, further comprising amplifying the plurality of barcoded nucleic acids to generate a plurality of double-stranded barcoded nucleic acids in the microwell using the single-stranded barcoded nucleic acids as templates.

72. The method of any one of claims 67-71, wherein the plurality of target nucleic acids comprises poly-adenylated messenger ribonucleic acid (mRNA) and the extension primers comprise a poly(dT) sequence.

73. The method of claim 71, wherein each of the plurality of barcode molecules comprises a primer sequence, optionally wherein the primer sequence comprises a PCR primer sequence, wherein amplifying the plurality of barcoded nucleic acids comprises amplifying the plurality of barcoded nucleic acids using the primer sequences in single-stranded barcoded nucleic acids of the plurality of single-stranded barcoded nucleic acids, or products thereof.

74. The method of any one of claims 33-73, wherein the plurality of target nucleic acids comprises deoxyribonucleic acid (DNA).

75. The method of any one of claims 33-74, wherein the plurality of target nucleic acids comprises ribonucleic acid (RNA).

76. The method of claim 75, wherein barcoding the plurality of target nucleic acids comprises a reverse transcription reaction, and wherein the plurality of barcoded nucleic acids comprises complementary deoxyribonucleic acid (cDNA).

77. The method of any one of claims 33-76, wherein barcoding the plurality of target nucleic acids comprises hybridizing the target binding sequence to a target nucleic acid of the plurality of target nucleic acids, and wherein the target binding sequence comprises a poly(dT) sequence and/or a sequence capable of hybridizing to the target nucleic acid, optionally wherein the sequence comprises a target specific sequence.

78. The method of any one of claims 33-77, wherein the target binding sequence of the barcode molecule comprises a poly(dT) sequence, and wherein barcoding the plurality of target nucleic acids comprises hybridizing the poly(dT) sequence of the target binding sequence to a poly(A) sequence of a target nucleic acid of the plurality of target nucleic acids

79. The method any one of claims 33-78, wherein releasing the plurality of target nucleic acids associated with the single cell in a microwell of the plurality of microwells comprises lysing the single cell using a lysis agent.

80. The method of claim 33-79, wherein the plurality of barcode molecules are attached to, reversibly attached to, covalently attached to, or irreversibly attached to the particle.

81. The method of any one of claims 33-80, wherein the particle is a bead.

82. The method of claim 81, wherein the particle is a gel particle, optionally wherein the gel particle is a hydrogel particle.

83. The method of claim 82, wherein the gel particle is degradable upon application of a stimulus.

84. The method of claim 83, wherein the stimulus comprises a thermal stimulus, a chemical stimulus, a biological stimulus, a photo-stimulus, or a combination thereof.

85. The method of claim 81, wherein the particle is a solid particle and/or a magnetic particle.

86. The method of claim 85, wherein the particle is retained in the microwell by an external magnetic field.

87. The method of claim 86, wherein the particle comprises a paramagnetic material.

88. The method of any one of claims 85-87, wherein the particle has a size of 10 μm to 100 μm.

89. The method any one of claims 61-88, wherein the molecular barcode sequence comprises unique molecule identifiers (UMIs).

90. The method of claim 89, wherein the UMIs are 2-40 nucleotides in length.

91. The method any one of claims 33-90 wherein the particle barcode sequences of the plurality of barcode molecules on a single particle are identical.

92. The method of any one of claims 33-91, wherein each of the plurality of barcode molecules comprises a primer sequence.

93. The method of claim 92, wherein the primer sequence is a sequencing primer sequence.

94. The method of claim 93, wherein the sequencing primer sequence is a Read 1 sequence, a Read 2 sequence, or a portion thereof.

95. The method of any one of claims 33-94, wherein a barcode molecule of the plurality of barcode molecules comprises a template switching oligonucleotide.

96. The method of any one of claims 33-95, wherein analyzing the plurality of barcoded nucleic acids, or products thereof, comprises determine the sequences of the plurality of barcoded nucleic acids, or products thereof.
